(19) United States
(12) Patent Application Publication
(10) Pub. No.: US 2023/0414760 A1
(43) Pub. Date: Dec. 28, 2023

(54) PHARMACEUTICAL FORMULATION

(71) Applicant: AstraZeneca UK Limited, Cambridge (GB)

(72) Inventors: Peng KE, Cambridge (GB); Maria Gabriela BRUQUE, Cambridge (GB); Katrina Jane DAY, Cambridge (GB)

(21) Appl. No.: 18/252,818

(22) PCT Filed: Nov. 11, 2021
(86) PCT No.: PCT/IB2021/060458

§ 371 Date: May 12, 2023

Related U.S. Application Data
(60) Provisional application No. 63/113,300, filed on Nov. 13, 2020.

(51) Int. Cl. A61K 47/22; A61K 47/68

(52) U.S. Cl. CPC A61K 47/22; A61K 47/68

(57) ABSTRACT

Described herein are stable pharmaceutical formulations for bioactive agents, including macromolecular bioactive agents such as polynucleotides and polypeptides. Also provided are medicaments and treatments using the pharmaceutical formulations described herein, as well as kits and methods of treating, preventing or managing a disease or disorder using the pharmaceutical formulations described herein.

### RELATED APPLICATIONS

This application claims the benefit of priority under 35 U.S.C. 119(e) to U.S. Provisional Application No. 63/113,300 filed on Nov. 13, 2020. The entire contents of the foregoing application are hereby incorporated by reference.

### FIELD OF THE INVENTION

The present disclosure relates to the field of pharmaceutical formulations. Specifically, a stable pharmaceutical formulation for biotherapeutics is provided.

### BACKGROUND

Biotherapeutics, which include macromolecule therapeutics, such as peptides, proteins, antibodies, polysaccharides, and nucleic acids; as well as cell-based therapeutics are a rapidly growing portion the therapeutics available to medical practitioners for treatment of a wide range of diseases and disorders. One reason for the success of biotherapeutics is the high specificity towards targets and superior safety profile as compared to small molecule therapeutics.

The structural complexity of biotherapeutics make them susceptible to physical or chemical degradation, which can result in structural and functional instability and reduced safety and efficacy. Although manufacturing and purification processes typically result in a high purity product, many biotherapeutics degrade over time, for example, during storage, transport and administration. Degradation can be due to chemical instability, which includes, but is not limited to, deamidation, isomerization, hydrolysis, racemization, beta-elimination, glycation, oxidation, adduct formation and disulfide scrambling; or physical instability, which includes, but is not limited to, denaturation, aggregation, precipitation, particle formation, and surface adsorption.

The composition of the pharmaceutical formulation for a biotherapeutic can significantly affect stability and, consequently, the safety and efficacy of the therapeutic. The pharmaceutical formulation also can affect the ease and frequency of administration and pain upon injection.

Surfactants are often included in pharmaceutical formulations to help stabilize the biotherapeutic and prevent aggregation or particle formation. Non-ionic surfactants such as polysorbate 80, polysorbate 20 and poloxamer 188 are commonly used in pharmaceutical formulations due to their proven safety profile.

However, polysorbate 80 and polysorbate 20 are known to be susceptible to degradation during storage by oxidative and hydrolytic pathways. Free fatty acids (FFA), one degradation product, can be sources of particle formation. Degradation can occur during storage of the polysorbate raw material, which can result in particle formation when the polysorbate surfactant is introduced into a pharmaceutical formulation. Degradation of the polysorbate surfactant in the pharmaceutical formulation can also result in aggregation. Additionally, degradation of polysorbates over time can result in an effective reduction in the amount of polysorbate in the formulations, adversely impacting the protective effect of the surfactant.

Despite advances made in the use of biotherapeutics and the knowledge relating to instability processes, there remains a need for pharmaceutical formulations with enhanced long-term stability. A formulation with improved long-term stability could help increase safety and efficacy of biotherapeutics. Thus, there remains a need for formulations that retain long-term stability under a variety of different manufacturing and storage conditions. The present invention satisfies this need and provides additional advantages as well.

### SUMMARY

A stable pharmaceutical formulation is provided herein that includes a bioactive agent and D-α-Tocopheryl polyethylene glycol succinate (TPGS) as a surfactant. In one aspect, the stable pharmaceutical formulation includes a bioactive agent and D-α-Tocopheryl polyethylene glycol 1000 succinate (TPGS 1000) as a surfactant. In one aspect, the formulation includes a buffer and has a pH from about 3 to about 9, about 4 to about 8, or about 5.5 to about 7.5.

In one aspect, the formulation is a liquid formulation. In one aspect, the formulation is an aqueous formulation. In one aspect, the formulation is lyophilized.

In one aspect, the bioactive agent is a biotherapeutic. In one aspect, the bioactive agent is a therapeutic macromolecule.

In one aspect, the bioactive agent includes a therapeutic polypeptide. In one aspect, the therapeutic polypeptide is selected from an antibody or antigen-binding antibody fragment, an enzyme or enzymatically-active polypeptide, a soluble receptor or receptor ligand, hormone, neurotransmitter, growth factor, integrin, interferon or an antigen. In one aspect, the antibody or antigen-binding antibody fragment includes a monoclonal antibody. In one aspect, the antibody or antigen-binding antibody fragment is selected from human, humanized, chimeric, multispecific, bispecific, Fab′, F(ab′)2, Fv, single chain Fv (scFv), diabodies, peptibodies, linear antibodies and single-chain antibodies. In one aspect, the therapeutic polypeptide includes a fusion polypeptide, a proteolysis targeting chimera (protac), or an antibody-drug conjugate (ADC).

In one aspect, the bioactive agent includes a therapeutic polynucleotide. In one aspect, the therapeutic polynucleotide includes single-stranded or double-stranded DNA or RNA. In one aspect, the therapeutic polynucleotide is selected from cDNA, antisense RNA, microRNA (miRNA), shorthairpin RNA (snRNA), RNA interference (RNAi), small interfering RNA (siRNA) and ribozymes. In one aspect, the therapeutic polynucleotide includes a plasmid, phagemid, cosmid or yeast artificial chromosomes (YAC). In one aspect, the therapeutic polynucleotide includes a viral vector, such as a retroviral, adenoviral, poxviral, adeno-associated viral (AAV), Newcastle disease viral (NDV) or herpes simplex viral vector.

In one aspect, the bioactive agent includes cells selected from stem cells, progenitor cells and differentiated cells.

In one aspect, the formulation includes the bioactive agent at a concentration from about mg/mL to about 300 mg/mL. In one aspect, the bioactive agent includes an antibody or antigen-binding antibody fragment with a concentration from about 10 mg/mL to about 200 mg/mL. In one aspect, the concentration of antibody or antigen-binding antibody fragment is at least about 10 mg/mL, about 15 mg/mL, about 20 mg/mL, about 25 mg/mL, about 50 mg/mL, about 75 mg/mL or about 100 mg/mL and up to about 125 mg/mL, about 150 mg/mL, about 175 mg/mL, about 200 mg/mL, about 250 mg/mL or about 300 mg/mL. In one aspect, the bioactive agent includes a therapeutic protein with a concentration from about 0.1 mg/mL, about 0.5 mg/mL, about 1 mg/mL, about 2 mg/mL, about 3 mg/mL, about 4 mg/mL or about 5 mg/mL and up to about 10 mg/mL, about 15 mg/mL, about 20 mg/mL, about 25 mg/mL, about 30 mg/mL, about 35 mg/mL, about 35 mg/mL, about 40 mg/mL, about 45 mg/mL or about 50 mg/mL.

In one aspect, the concentration of TPGS in the pharmaceutical formulation is from about to about 1%. In one aspect, the concentration of TPGS is from about 0.001%, about about 0.01%, about 0.05% and up to about 0.1%, about 0.5% or about 1%. In one aspect, the concentration of TPGS is about 0.01%, about 0.02%, about 0.03%, about 0.04%, about 0.05%, about 0.06%, about 0.07%, about 0.08%, about 0.09% or about 0.1%. In one aspect, the concentration of TPGS is about 0.01%. In one aspect, the concentration of TPGS is about 0.02%. In one aspect, the concentration of TPGS is about 0.03%. In one aspect, the concentration of TPGS is about 0.04%. In one aspect, the concentration of TPGS is about 0.05%. In one aspect, the concentration of TPGS is about 0.06%. In one aspect, the concentration of TPGS is about 0.07%. In one aspect, the concentration of TPGS is about 0.08%. In one aspect, the concentration of TPGS is about 0.09%. In one aspect, the concentration of TPGS is about 0.1%.

In one aspect, the pharmaceutical formulation includes a buffer. In one aspect, the buffer is selected from acetate, acetic acid, succinate, succinic acid, phosphate, phosphoric acid, ascorbate, ascorbic acid, lactate, lactic acid, tartartic acid, maleic acid, glycine, gluconate, citrate, histidine, imidazole, bicarbonate and carbonic acid, sodium benzoate, benzoic acid, edetate, malate, tris, glycylglycine and mixtures thereof. In one aspect, the buffer includes histidine/histidine hydrochloride (HCl). In one aspect, the buffer includes tris/tris hydrochloride (Tris HCl). In one aspect, the buffer includes citrate. In one aspect, the buffer includes sodium acetate. In one aspect, the buffer includes phosphate. In one aspect, the buffer has a concentration from about 0.1 mM to about 100 mM. In one aspect, the buffer concentration is from about 0.1 mM, about 0.5 mM, about 1 mM, about 5 mM, about 10 mM, about 20 mM or about 25 mM and up to about 30 mM, about 35 mM, about 40 mM, about 45 mM, or about 50 mM. In one aspect, the buffer includes about 5 mM histidine/histidine HCl. In one aspect, the buffer includes about 10 mM histidine/histidine HCl. In one aspect, the buffer includes about 15 mM histidine/histidine HCl. In one aspect, the buffer includes about 20 mM histidine/histidine HCl. In one aspect, the buffer includes about 25 mM histidine/histidine HCl. In one aspect, the buffer includes about 30 mM histidine/histidine HCl.

In one aspect, the formulation includes a tonicity agent. In one aspect, the tonicity agent includes a polyol, a saccharide, a carbohydrate, a salt, or a mixture thereof. In one aspect, the formulation includes the tonicity reagent at a concentration from about 1 mg/ml to about 300 mg/ml, about 10 mg/ml to about 200 mg/ml or about 50 mg/ml to about 100 mg/ml. In one aspect, the tonicity reagent includes a saccharide at a concentration of about 80 mg/ml to about 90 mg/ml. In one aspect, the tonicity reagent includes a salt at a concentration from about 1 mg/mL to about 20 mg/ml.

In one aspect, the formulation includes an amino acid as an excipient. In one aspect, the amino acid is selected from arginine, cysteine, glycine, lysine, ornithine, proline, alanine, glutamine, glutamic acid, histidine, valine or a combination thereof. In one aspect, the formulation includes an amino acid salt as an excipient. In one aspect, the formulation includes an amino acid salt selected from a salt form of arginine, cysteine, glycine, lysine, ornithine, proline, alanine, glutamine, glutamic acid, histidine, valine or a combination thereof. In one aspect, the formulation includes a chelating agent. In one aspect, the chelating agent is selected from aminopolycarboxylic acids, hydroxyaminocarboxylic acids, N-substituted glycines, 2-(2-amino-2-oxocthyl) aminoethane sulfonic acid (BES), deferoxamine (DEF), citric acid, niacinamide, and desoxycholates and mixtures thereof. In one aspect, the chelating agent is selected from ethylenediaminetetraacetic acid (EDTA), diethylenetriamine pentaacetic acid (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid (HIMDA), N,N-bis-hydroxyethylglycine (bicine) and N-(trishydroxymethylmethyl) glycine (tricine), glycylglycine, sodium desoxycholate, ethylenediamine; propylenediamine; diethylenetriamine; triethylenetetraamine (trien), ethylenediaminetetraaceto EDTA; disodium EDTA, calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In one aspect, the concentration of the chelating agent is from about 0.01 mg/ml to about 50 mg/ml.

In one aspect, the stable pharmaceutical formulation has less than about 10,000, about 5,000 about 1,000, about 750, about 500, about 250, about 150, about 100, or about 50 particles greater than about 2 μm, about 5 μm, about 10 μm, about 15 μm, about 20 μm or about 25 μm diameter/mL.

In one aspect, the formulation is stable at a temperature of about 40° C. for up to 3 months. In one aspect, the formulation is stable at a temperature of about 40° C. for up to 6 months. In one aspect, the formulation is stable at a temperature of about 25° C. for up to 6 months. In one aspect, the formulation is stable at a temperature of about 25° C. for up to 12 months. In one aspect, the formulation is stable at a temperature of about 2° C. to about 8° C. for up to 12 months. In one aspect, the formulation is stable at a temperature of about 2° C. to about 8° C. for up to 24 months. In one aspect, the formulation is stable at a temperature of about 2° C. to about 8° C. for up to 36 months. In one aspect, the formulation is stable at a temperature of about −20° C. for up to 6 months. In one aspect, the formulation is stable at a temperature of about −20° C. for up to 12 months. In one aspect, the formulation is stable at a temperature of about −20° C. for up to 24 months. In one aspect, the formulation is stable at a temperature of about −20° C. for up to 36 months. In one aspect, the formulation is stable at a temperature of about −80° C. for up to 6 months. In one aspect, the formulation is stable at a temperature of about −80° C. for up to 12 months. In one aspect, the formulation is stable at a temperature of about −80° C. for up to 24 months. In one aspect, the formulation is stable at a temperature of about −80° C. for up to 36 months.

In one aspect, a method of reducing particle formation in an aqueous pharmaceutical formulation is provided. In one aspect, the method includes adding D-α-Tocopheryl polyethylene glycol succinate (TPGS) to the formulation. In one aspect, the method includes adding D-α-Tocopheryl polyethylene glycol 1000 succinate (TPGS 1000) to the formulation. In one aspect, the method includes adding D-α-Tocopheryl polyethylene glycol 1000 succinate (TPGS 1000) to the formulation in an amount ranging from about 0.001% to about 1%. In one aspect, less than about 10,000, about 5,000, about 1,000, about 750, about 500, about 250, about 150, about 100, or about 50 particles greater than about 2 μm, about 5 μm, about 10 μm, about 15 μm, about 20 μm or about 25 μm diameter/mL are formed. In one aspect, the pharmaceutical formulation includes less than about 5,000 particles greater than about 2 μm in diameter/mL. In one aspect, the pharmaceutical formulation includes less than about 1,000 particles greater than about 2 μm in diameter/mL. In one aspect, the pharmaceutical formulation includes less than about 6,000 particles greater than about 10 μm in diameter/mL. In one aspect, the pharmaceutical formulation includes less than about 600 particles greater than about 25 μm in diameter/mL.

In one aspect, a method of reducing interference during antibody-drug (ADC) conjugation is provided. In one aspect, the method includes adding D-α-Tocopheryl polyethylene glycol succinate (TPGS) to a formulation comprising an unconjugated antibody intermediate. In one aspect, D-α-Tocopheryl polyethylene glycol 1000 succinate (TPGS 1000) is added to the formulation in an amount ranging from about 0.001% to about 1%.

### BRIEF DESCRIPTION OF THE DRAWINGS

FIG. 1 is a schematic of the structure of D-α-Tocopheryl polyethylene glycol succinate (TPGS).

FIG. 2A is a graph showing the number of particles/ml with a diameter greater than or equal to 1 μm and less than 2 μm for four test molecules (mAb-1, mAb-2, mAb-3 and mAb-4) formulated with TPGS, Kolliphor HS15, Kolliphor EL, Kolliphor RH40, PEG 300, PS80, P188, or no surfactant when assayed for stability after shipping simulation (SS) using micro-flow imaging (MFI).

FIG. 2B is a graph showing the number of particles/ml with a diameter greater than or equal to 2 μm for four test molecules (mAb-1, mAb-2, mAb-3 and mAb-4) formulated with TPGS, Kolliphor HS15, Kolliphor EL, Kolliphor RH40, PEG 300, PS80, P188, or no surfactant when assayed for stability after shipping simulation (SS) using micro-flow imaging (MFI).

FIG. 2C is a graph showing the number of particles/ml with a diameter greater than or equal to 10 μm for four test molecules (mAb-1, mAb-2, mAb-3 and mAb-4) formulated with TPGS, Kolliphor HS15, Kolliphor EL, Kolliphor RH40, PEG 300, PS80, P188, or no surfactant when assayed for stability after shipping simulation (SS) using micro-flow imaging (MFI).

FIG. 2D is a graph showing the number of particles/ml with a diameter greater than or equal to 25 μm for four test molecules (mAb-1, mAb-2, mAb-3 and mAb-4) formulated with TPGS, Kolliphor HS15, Kolliphor EL, Kolliphor RH40, PEG 300, PS80, P188, or no surfactant when assayed for stability after shipping simulation (SS) using micro-flow imaging (MFI).

FIG. 3 is a graph showing the % monomer for four test molecules (mAb-1, mAb-2, mAb-4 and mAb-3) formulated with TPGS, Kolliphor HS15, Kolliphor EL, Kolliphor RH40, PEG 300, PS80, P188, or no surfactant and assayed for stability after shipping simulation (SS) using high pressure size exclusion chromatography (HPSEC).

FIG. 4A is a graph showing the number of particles/ml with a diameter greater than or equal to 1 μm and less than 2 μm for four test molecules (mAb-1, mAb-2, mAb-3 and mAb-4) formulated with TPGS, PS80, and P188 and stored at 40° C. for 4 weeks as determined by micro-flow imaging (MFI).

FIG. 4B is a graph showing the number of particles/ml with a diameter greater than or equal to 2 μm for four test molecules (mAb-1, mAb-2, mAb-3 and mAb-4) formulated with TPGS, PS80, and P188 and stored at 40° C. for 4 weeks as determined by micro-flow imaging (MFI).

FIG. 4C is a graph showing the number of particles/ml with a diameter greater than or equal to 10 μm for four test molecules (mAb-1, mAb-2, mAb-3 and mAb-4) formulated with TPGS, PS80, and P188 and stored at 40° C. for 4 weeks as determined by micro-flow imaging (MFI).

FIG. 4D is a graph showing the number of particles/ml with a diameter greater than or equal to 25 μm for four test molecules (mAb-1, mAb-2, mAb-3 and mAb-4) formulated with TPGS, PS80, and P188 and stored at 40° C. for 4 weeks as determined by micro-flow imaging (MFI).

FIG. 5A is a graph showing the % monomer for mAb-1 formulated with TPGS, PS80, and P188 and stored at 40° C. for 0-4 weeks. Stability was evaluated by determining % monomer using high pressure size exclusion chromatography (HPSEC).

FIG. 5B is a graph showing the % monomer for mAb-2 formulated with TPGS, PS80, and P188 and stored at 40° C. for 0-4 weeks. Stability was evaluated by determining % monomer using high pressure size exclusion chromatography (HPSEC).

FIG. 5C is a graph showing the % monomer for mAb-3 formulated with TPGS, PS80, and P188 and stored at 40° C. for 0-4 weeks. Stability was evaluated by determining % monomer using high pressure size exclusion chromatography (HPSEC).

FIG. 5D is a graph showing the % monomer for mAb-4 formulated with TPGS, PS80, and P188 and stored at 40° C. for 0-4 weeks. Stability was evaluated by determining % monomer using high pressure size exclusion chromatography (HPSEC).

FIG. 6A is a graph showing the number of particles/ml with a diameter greater than or equal to 1 μm and less than 2 μm for four test molecules (mAb-1, mAb-2, mAb-3 and mAb-4) formulated with TPGS, PS80, and P188 and stored at 25° C. for 6 months as determined by micro-flow imaging (MFI).

FIG. 6B is a graph showing the number of particles/ml with a diameter greater than or equal to 2 μm for four test molecules (mAb-1, mAb-2, mAb-3 and mAb-4) formulated with TPGS, PS80, and P188 and stored at 25° C. for 6 months as determined by micro-flow imaging (MFI).

FIG. 6C is a graph showing the number of particles/ml s with a diameter greater than or equal to 10 μm for four test molecules (mAb-1, mAb-2, mAb-3 and mAb-4) formulated with TPGS, PS80, and P188 and stored at 25° C. for 6 months as determined by micro-flow imaging (MFI).

FIG. 6D is a graph showing the number of particles/ml with a diameter greater than or equal to 25 μm for four test molecules (mAb-1, mAb-2, mAb-3 and mAb-4) formulated with TPGS, PS80, and P188 and stored at 25° C. for 6 months as determined by micro-flow imaging (MFI).

FIG. 7A is a graph showing the % monomer for mAb-1 formulated with TPGS, PS80, and P188 and stored at 25° C. for 0-6 months. Stability was evaluated by determining % monomer using high pressure size exclusion chromatography (HPSEC).

FIG. 7B is a graph showing the % monomer for mAb-2 formulated with TPGS, PS80, and P188 and stored at 25° C. for 0-6 months. Stability was evaluated by determining % monomer using high pressure size exclusion chromatography (HPSEC).

FIG. 7C is a graph showing the % monomer for mAb-3 formulated with TPGS, PS80, and P188 and stored at 25° C. for 0-6 months. Stability was evaluated by determining % monomer using high pressure size exclusion chromatography (HPSEC).

FIG. 7D is a graph showing the % monomer for mAb-4 formulated with TPGS, PS80, and P188 and stored at 25° C. for 0-6 months. Stability was evaluated by determining % monomer using high pressure size exclusion chromatography (HPSEC).

FIG. 8A is a graph showing the number of particles/ml with a diameter greater than or equal to 1 μm and less than 2 μm for four test molecules (mAb-1, mAb-2, mAb-3 and mAb-4) formulated with TPGS, PS80, and P188 and stored at 5° C. for 6 months as determined by micro-flow imaging (MFI).

FIG. 8B is a graph showing the number of particles/ml with a diameter greater than or equal to 2 μm for four test molecules (mAb-1, mAb-2, mAb-3 and mAb-4) formulated with TPGS, PS80, and P188 and stored at 5° C. for 6 months as determined by micro-flow imaging (MFI).

FIG. 8C is a graph showing the number of particles/ml s with a diameter greater than or equal to 10 μm for four test molecules (mAb-1 mAb-2, mAb-3 and mAb-4) formulated with TPGS, PS80, and P188 and stored at 5° C. for 6 months as determined by micro-flow imaging (MFI).

FIG. 8D is a graph showing the number of particles/ml with a diameter greater than or equal to 25 μm for four test molecules (mAb-1, mAb-2, mAb-3 and mAb-4) formulated with TPGS, PS80, and P188 and stored at 5° C. for 6 months as determined by micro-flow imaging (MFI).

FIG. 9A is a graph showing the % monomer for mAb-1 formulated with TPGS, PS80, and P188 and stored at 5° C. for 0-6 months. Stability was evaluated by determining % monomer using high pressure size exclusion chromatography (HPSEC).

FIG. 9B is a graph showing the % monomer for mAb-2 formulated with TPGS, PS80, and P188 and stored at 5° C. for 0-6 months. Stability was evaluated by determining % monomer using high pressure size exclusion chromatography (HPSEC).

FIG. 9C is a graph showing the % monomer for mAb-3 formulated with TPGS, PS80, and P188 and stored at 5° C. for 0-6 months. Stability was evaluated by determining % monomer using high pressure size exclusion chromatography (HPSEC).

FIG. 9D is a graph showing the % monomer for mAb-4 formulated with TPGS, PS80, and P188 and stored at 5° C. for 0-6 months. Stability was evaluated by determining % monomer using high pressure size exclusion chromatography (HPSEC).

FIG. 10A is a graph showing the number of particles/ml with a diameter greater than or equal to 1 μm and less than 2 μm for four test molecules (mAb-1, mAb-2, mAb-3 and mAb-4) formulated with TPGS, PS80, and P188 and stored at −80° C. for 6 months as determined by micro-flow imaging (MFI).

FIG. 10B is a graph showing the number of particles/ml with a diameter greater than or equal to 2 μm for four test molecules (mAb-1, mAb-2, mAb-3 and mAb-4) formulated with TPGS, PS80, and P188 and stored at −80° C. for 6 months as determined by micro-flow imaging (MFI).

FIG. 10C is a graph showing the number of particles/ml with a diameter greater than or equal to 10 μm for four test molecules (mAb-1, mAb-2, mAb-3 and mAb-4) formulated with TPGS, PS80, and P188 and stored at −80° C. for 6 months as determined by micro-flow imaging (MFI).

FIG. 10D is a graph showing the number of particles/ml with a diameter greater than or equal to 25 μm for four test molecules (mAb-1, mAb-2, mAb-3 and mAb-4) formulated with TPGS, PS80, and P188 and stored at −80° C. for 6 months as determined by micro-flow imaging (MFI).

FIG. 11A is a graph showing the % monomer for mAb-1 formulated with TPGS, PS80, and P188 and stored at −80° C. for 0-6 months. Stability was evaluated by determining % monomer using high pressure size exclusion chromatography (HPSEC).

FIG. 11B is a graph showing the % monomer for mAb-2 formulated with TPGS, PS80, and P188 and stored at −80° C. for 0-6 months. Stability was evaluated by determining % monomer using high pressure size exclusion chromatography (HPSEC).

FIG. 11C is a graph showing the % monomer for mAb-3 formulated with TPGS, PS80, and P188 and stored at −80° C. for 0-6 months. Stability was evaluated by determining % monomer using high pressure size exclusion chromatography (HPSEC).

FIG. 11D is a graph showing the % monomer for mAb-4 formulated with TPGS, PS80, and P188 and stored at −80° C. for 0-6 months. Stability was evaluated by determining % monomer using high pressure size exclusion chromatography (HPSEC).

FIG. 12A is a graph showing the number of particles/ml with a diameter greater than or equal to 1 μm and less than 2 μm for mAb-2 formulated with varying concentrations of TPGS, PS80, PS20 or P188 and subjected to a shipping simulation (SS). Stability was assessed via micro-flow imaging (MFI).

FIG. 12B is a graph showing the number of particles/ml with a diameter greater than or equal to 2 μm for mAb-2 formulated with varying concentrations of TPGS, PS80, PS20 or P188 and subjected to a shipping simulation (SS). Stability was assessed via micro-flow imaging (MFI).

FIG. 12C is a graph showing the number of particles/ml with a diameter greater than or equal to 10 μm for mAb-2 formulated with varying concentrations of TPGS, PS80, PS20 or P188 and subjected to a shipping simulation (SS). Stability was assessed via micro-flow imaging (MFI).

FIG. 12D is a graph showing the number of particles/ml with a diameter greater than or equal to 25 μm for mAb-2 formulated with varying concentrations of TPGS, PS80, PS20 or P188 and subjected to a shipping simulation (SS). Stability was assessed via micro-flow imaging (MFI).

FIG. 13 is a table showing stability of mAb-2 formulated with varying concentrations of TPGS, PS80, PS20 or P188 and subjected to a shipping simulation (SS). Stability was assessed via high-pressure size exclusion chromatography (HPSEC) and evaluated for % monomer (% Mon), % high molecular weight species (% HMW), and % fragmentation (% Frag). *The apparent increase in % HMW observed for the sample containing 1.00% TPGS was due to interreference of TPGS with the HPSEC column, rather than aggregation of the molecule.

FIG. 14 shows overlaid HPSEC chromatograms of blank buffer containing different levels of TPGS. At 1.0% TPGS concentration, the area of the peak increases which results in interference with the mAb chromatograms. At the lower concentrations, the interreference of TPGS is negligible. This behaviour is found with other surfactants such as PS80.

FIG. 15 shows overlaid HPSEC chromatograms of blank buffer containing 1.0% TPGS with mAb-2 samples containing 0.2% or 1.0% TPGS. The TPGS eluted at a similar retention time as the aggregate peak of mAb-2 demonstrating that inclusion of 1.0% TPGS can result in an overestimation of aggregate level.

FIG. 16 is a table showing capillary gel electrophoresis (CGE) results for formulations of mAb-2 containing PS80, PS20, Poloxamer 188 and different concentrations of TPGS. CGE is not subject to interference from surfactants and the samples containing 1.00% TPGS had comparable results with other sample conditions.

FIG. 17A is a graph showing the number of particles/ml with a diameter greater than or equal to 1 μm and less than 2 μm for mAb-4 formulated with varying concentrations of TPGS, PS80, PS20 or P188 and subjected to a shipping simulation (SS). Stability was assessed via micro-flow imaging (MFI).

FIG. 17B is a graph showing the number of particles/ml with a diameter greater than or equal to 2 μm for mAb-4 formulated with varying concentrations of TPGS, PS80, PS20 or P188 and subjected to a shipping simulation (SS). Stability was assessed via micro-flow imaging (MFI).

FIG. 17C is a graph showing the number of particles/ml with a diameter greater than or equal to 10 μm for mAb-4 formulated with varying concentrations of TPGS, PS80, PS20 or P188 and subjected to a shipping simulation (SS). Stability was assessed via micro-flow imaging (MFI).

FIG. 17D is a graph showing the number of particles/ml with a diameter greater than or equal to 25 μm for mAb-4 formulated with varying concentrations of TPGS, PS80, PS20 or P188 and subjected to a shipping simulation (SS). Stability was assessed via micro-flow imaging (MFI).

FIG. 18 is a table showing stability of mAb-4 formulated with varying concentrations of TPGS, PS80, PS20 or P188 and subjected to a shipping simulation (SS). Stability was assessed via high-pressure size exclusion chromatography (HPSEC) and evaluated for % monomer (% Mon), % high molecular weight species (% HMW), and % fragmentation (% Frag). *The apparent increase in % HMW observed for the sample containing 1.00% TPGS was due to interreference of TPGS with the HPSEC column, rather than aggregation of the molecule.

FIG. 19 is a table showing capillary gel electrophoresis (CGE) results for formulations of mAb-4 containing PS80, PS20, Poloxamer 188 and different concentrations of TPGS. CGE is not subject to interference from surfactants and the samples containing 1.00% TPGS had comparable results with other sample conditions.

FIG. 20A is a table showing stability of mAb-5 formulated with TPGS or control. Stability was assessed at time zero (T0), after 3 freeze-thaw cycles (3-FT) and post-shipping simulation (SS) via micro-flow imaging (MFI).

FIG. 20B is a table showing stability of mAb-6 formulated with TPGS or control. Stability was assessed at time zero (T0), after 3 freeze-thaw cycles (3-FT) and post-shipping simulation (SS) via micro-flow imaging (MFI).

FIG. 21A is a graph showing the number of particles/ml with a diameter greater than or equal to 1 μm and less than 2 μm for mAb-5 formulated with TPGS or control and subjected to a shipping simulation (SS). Stability was assessed at time zero (T0), after 3 freeze-thaw cycles (3-FT), via micro-flow imaging (MFI).

FIG. 21B is a graph showing the number of particles/ml with a diameter greater than or equal to 2 μm for mAb-5 formulated with TPGS or control and subjected to a shipping simulation (SS). Stability was assessed at time zero (T0), after 3 freeze-thaw cycles (3-FT), via micro-flow imaging (MFI).

FIG. 21C is a graph showing the number of particles/ml with a diameter greater than or equal to 10 μm for mAb-5 formulated with TPGS or control and subjected to a shipping simulation (SS). Stability was assessed at time zero (T0), after 3 freeze-thaw cycles (3-FT), via micro-flow imaging (MFI).

FIG. 21D is a graph showing the number of particles/ml with a diameter greater than or equal to 25 μm for mAb-5 formulated with TPGS or control and subjected to a shipping simulation (SS). Stability was assessed at time zero (T0), after 3 freeze-thaw cycles (3-FT), via micro-flow imaging (MFI).

FIG. 22A is a graph showing the number of particles/ml with a diameter greater than or equal to 1 μm and less than 2 μm for mAb-6 formulated with TPGS or control and subjected to a shipping simulation (SS). Stability was assessed at time zero (T0), after 3 freeze-thaw cycles (3-FT), via micro-flow imaging (MFI).

FIG. 22B is a graph showing the number of particles/ml with a diameter greater than or equal to 2 μm for mAb-6 formulated with TPGS or control and subjected to a shipping simulation (SS). Stability was assessed at time zero (T0), after 3 freeze-thaw cycles (3-FT), via micro-flow imaging (MFI).

FIG. 22C is a graph showing the number of particles/ml with a diameter greater than or equal to 10 μm for mAb-6 formulated with TPGS or control and subjected to a shipping simulation (SS). Stability was assessed at time zero (T0), after 3 freeze-thaw cycles (3-FT), via micro-flow imaging (MFI).

FIG. 22D is a graph showing the number of particles/ml with a diameter greater than or equal to 25 μm for mAb-6 formulated with TPGS or control and subjected to a shipping simulation (SS). Stability was assessed at time zero (T0), after 3 freeze-thaw cycles (3-FT), via micro-flow imaging (MFI).

FIG. 23 is a table showing stability of mAb-5 and mAb-6 formulated with TPGS or control and subjected to a shipping simulation (SS). Stability was assessed stability at time zero (T0), after 3 freeze-thaw cycles (3-FT), and post-shipping simulation (SS) via high-pressure size exclusion chromatography (HPSEC).

### DETAILED DESCRIPTION

### Definitions

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, “a” or “an” may mean one or more. As used herein in the specification and claims, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the disclosure and claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, or composition of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

The term “pharmaceutical formulation” refers to a composition that includes a bioactive agent and a pharmaceutically acceptable carrier, excipient, diluent or other pharmaceutically acceptable additive. The formulation can be suitable for diagnostic, therapeutic, or preventive use. The formulation can be suitable for use in vitro, in vivo, or ex vivo. In one aspect, the pharmaceutical formulation is administered parenterally, including, but not limited to, intravenously, intramuscularly or subcutaneously. Pharmaceutical formulations can include liquid formulations, for example, aqueous solutions, or lyophilized powders that can be reconstituted into a suitable solution for administration, for example, by adding an appropriate diluent before administration.

A “stable” formulation refers to a formulation in which the bioactive agent retains its physical stability, chemical stability or biological activity during storage. “Chemical stability” can be assessed by detecting and quantifying chemically altered forms of the bioactive agent, including, for example, deamidation, including, for example, asparagine (Asn) deamidation; isomerization, including, for example, aspartate (Asp) isomerization; oxidation, including for example methionine (Met) oxidation; clipping/hydrolysis/fragmentation, including, for example, antibody hinge region fragmentation; succinimide formation; racemization; beta-elimination; glycation; adduct formation; disulfide scrambling; N-terminal extension; C-terminal processing; and glycosylation differences. “Physical stability” can be assessed by detecting and quantifying physically altered forms of the bioactive agent, including, but not limited to, physically altered forms due to denaturation, aggregation, precipitation or particle formation, and surface adsorption. The term “biological activity” refers to a function of a molecule and can encompass, biological function, biochemical function, physical function, and chemical function. Examples of biological activity include, but are not limited to, enzymatic activity; ability to interact with or bind to another molecule; ability to activate, promote, stabilize, inhibit, suppress, or destabilize a function of another molecule; and ability to localize to a specific position in a cell. As used herein, “biological function,” with regard to a polynucleotide, for example, a gene or polypeptide related thereto, can refer to a specific function that the polynucleotide, gene, or polypeptide can have in a living body. Examples include, but are not limited to, production of a specific protein, enzymatic activity, impartation of resistance, and the like. Biological activity can be measured using technique known in the art. “Improved stability” or “increased stability” refers to a pharmaceutical formulation in which the physical stability, chemical stability or biological activity of the bioactive agent is qualitatively or quantitatively evaluated and which is increased in comparison to the physical stability, chemical stability or biological activity of a reference composition of the bioactive agent.

Stability can be evaluated qualitatively or quantitatively in a variety of different ways, including, but not limited to, evaluation of aggregate formation (for example using size exclusion chromatography, by measuring turbidity, or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE; peptide map analysis; or by evaluating biological activity, for example, via an in vitro, in vivo or in situ assay indicative of bioactive agent activity.

In one aspect, a bioactive agent is “stable” in a pharmaceutical formulation, if the physical stability, chemical stability or biological activity of the bioactive agent at a given time is within about 0.1%, about 0.5%, about 1%, about 5%, about 10%, about 15%, about 20% or about 25% (within a standard of error) of the physical stability, chemical stability or biological activity of the bioactive agent exhibited at an initial time point, for example, at the time the pharmaceutical formulation was prepared.

The term “bioactive agent” can refer to any substance that is suitable for therapeutic, prophylactic, or diagnostic use and can be used interchangeably with the term “therapeutic agent.” A bioactive agent can include “biotherapeutic” products in which the active substance is obtained from a biological source. Examples of biotherapeutics include macromolecules, for example, therapeutic polynucleotides or polypeptides; and cell-based therapeutics. Examples of biotherapeutics include, but are not limited to recombinant proteins and hormones, monoclonal antibodies (mAbs), cytokines, growth factors, gene therapy products, vaccines, gene-silencing/editing therapies, cell-based therapeutics, tissue-engineered products, and stem cell therapies. Bioactive agents include, but are not limited to, naturally occurring or recombinantly produced cells or macromolecules, including polypeptides and polynucleotides; and cells, including, for example, stem cells, progenitor cells or differentiated cells. The term “polypeptide” and “protein” are used interchangeably to refer to a molecule having two or more amino acid residues joined to each other by peptide bonds and includes antibodies, antigen-binding antibody fragments, non-antibody binding proteins, therapeutic proteins and therapeutic peptides. In one aspect, the bioactive agent is of therapeutic, scientific or commercial interest.

A “therapeutically effective amount” of a bioactive agent refers to an amount effective in the prevention or treatment of a disease or disorder in a subject. A “therapeutically effective amount” can vary depending on factors such as disease state, age, sex, and weight of subject. In one aspect, a “therapeutically effective amount” refers to an amount of the bioactive agent sufficient to ameliorate at least one symptom associated with a disease or disorder. In one aspect, a therapeutically effective amount encompasses an amount in which any toxic or detrimental effects of the bioactive agent are outweighed by the therapeutically beneficial effects. A “therapeutically effective amount” can be administered in one or more administrations.

“Subject” can refer to any subject in need of treatment or that is at risk for, or is predisposed to, developing a pathologic condition. The term “subject” includes mammals, such as humans, dogs, cows, horses, pigs, sheep, goats, cats, mice, rabbits, rats, and transgenic non-human animals. In one aspect, the subject is human.

“Pharmaceutically acceptable” means that a compound can safely be administered to a subject to provide an effective dose of the bioactive agent.

The term “recombinant” when used in reference to a polynucleotide, peptide, polypeptide, protein or cell means of, or resulting from, a new combination of material that is not known to exist in nature.

“Excipient” refers to a therapeutically inactive substance in a pharmaceutical formulation. Excipients can be included in a formulation for a wide variety of purposes, including, for example, as a diluent, vehicle, buffer, stabilizer, tonicity agent, bulking agent, surfactant, cryoprotectant, lyoprotectant, anti-oxidant, metal ion source, chelating agent or preservative.

“Amino acid salt” refers to a cationic or anionic form of an amino acid in combination with a counter ion with an opposite charge. In one aspect, the amino acid salt is a pharmacologically acceptable salt. In one aspect, the amino acid salt is an inorganic salt. In one aspect, the amino acid salt is an organic salt. In one aspect, the amino acid salt includes a sodium salt, potassium salt, calcium salt, magnesium salt, ammonium salt, hydrochloride salt, sulfate salt, nitrate salt, or phosphate salt. In one aspect, the amino acid salt includes an organic acid salt such as acetate, citrate, maleate, malate, or oxalate salt. In one aspect, the formulation includes an amino acid salt selected from a salt form of arginine, cysteine, glycine, lysine, ornithine, proline, alanine, glutamine, glutamic acid, histidine, valine or a combination thereof. In one aspect, the amino acid salt includes arginine, lysine or histidine.

“Surfactant” refers to a surface-active agent that lowers the surface tension of a liquid in which it is dissolved. Surfactants can be included in a pharmaceutical formulation for a variety of reasons including, for example, to prevent or control aggregation, particle formation or surface adsorption in liquid formulations or to prevent or control these phenomena during lyophilization or reconstitution of lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and to form micelles. Surfactants can be anionic, non-ionic, cationic, amphoteric, zwitterionic, and combinations thereof. In one aspect, the surfactant includes vitamin E (alpha-tocopherol) polyethylene glycol succinate (TPGS). In one aspect, the surfactant includes D-α-tocopheryl polyethylene glycol 1000 succinate (TPGS 1000).

As used herein, “buffer” refers to a composition that resists changes in pH due to the presence of acid-base conjugate components. Examples of buffers include, but are not limited to, acetate, acetic acid, succinate, succinic acid, phosphate, phosphoric acid, ascorbate, ascorbic acid, lactate, lactic acid, tartartic acid, maleic acid, glycine, gluconate, citrate, histidine, imidazole, bicarbonate and carbonic acid, sodium benzoate, benzoic acid, edetate, malate, tris, glycylglycine and mixtures thereof. In one aspect, the pharmaceutical formulation has a pH in the range from about 3.0 to about 9.0, from about 4.0 to about 8.0, or from about 5.5 to about 7.5, or at least about 3.0, about 3.5, about 4.0, about 4.5, about 5.0, about 5.5, about 6.0, about 6.5 or about 7.0 and up to about 7.0, about 7.5, about 8.0, about 8.5, or about 9.0. In one aspect, the pharmaceutical formulation has a pH of about 3.0, about 3.5, about 4.0, about 4.5, about 5.0, about 5.5, about 6.0, about 6.5, about 7.0, about 7.5, about 8.0, about 8.5, or about 9.0.

“Tonicity agent” or “tonicifier” refers to an excipient that can adjust the osmotic pressure of a pharmaceutical formulation. In one aspect, the tonicity agent is used to adjust the osmotic pressure of a pharmaceutical formulation to isotonic so that the formulation is physiologically compatible with cells and tissues of the subject. In one aspect, an “isotonic” formulation is one that has a similar osmotic pressure as human blood, generally from about 250 mOsm to about 350 mOsm. “Hypotonic” describes a formulation with an osmotic pressure below that of human blood and “hypertonic” describes a formulation with an osmotic pressure above that of human blood. Methods for determining isotonicity are known and include, for example, measuring a vapor pressure or use of an ice-freezing type osmometer. In one aspect, the “tonicity agent” improves the stability of a therapeutic macromolecule in a pharmaceutical formulation.

A “chelating agent” is a chemical compound capable of forming a complex with a metal ion. Generally, chelating agents include organic compounds that include a ring-like center capable of forming bonds with the metal atom and are included in pharmaceutical formulations to convert metal ions in the formulation to a chemically inert form.

In one aspect, the pharmaceutical formulation includes an amino acid excipient. In one aspect, the pharmaceutical formulation includes an amino acid salt as an excipient. In one aspect, the pharmaceutical formulation includes an amino acid excipient as an aggregation inhibitor. While not wishing to be bound by theory, it is believed that inclusion of one or more amino acids as an excipient in a pharmaceutical formulation can stabilize a proteinaceous bioactive agent by mechanisms such as preferential hydration or by direct interactions between the protein and ions.

The term “diluent” refers to a pharmaceutically acceptable solvent that can be used to prepare a liquid pharmaceutical formulation and includes, but is not limited to, sterile water and bacteriostatic water for injection (BWFI).

### Overview

Provided herein are stable pharmaceutical formulations for bioactive agents. In one aspect, the bioactive agent is a biotherapeutic. In one aspect, the biotherapeutic is a therapeutic macromolecule, such as a therapeutic polynucleotide or a therapeutic protein. In one aspect, the biotherapeutic includes cells. Long-term stability of a biotherapeutic can impact safety, consistency and efficacy of treatment. Loss of functionality within a preparation can decrease the effective concentration of the therapeutic for a given administration. Similarly, undesired modifications of a biotherapeutic can affect the activity or the safety of a formulation, which can result in loss of efficacy and increased risk of adverse side effects.

Non-ionic surfactants such as polysorbate 80, polysorbate 20 and poloxamer 188 are widely used in biotherapeutic formulations to protect against aggregation and particle formation. However, polysorbate 80 and polysorbate 20 are known to be susceptible to degradation during storage by oxidative and hydrolytic pathways, which can result in aggregation and particle formation when included in a pharmaceutical formulation. Additionally, these non-ionic surfactants are not easily removed from formulations, for example, using processes such as diafiltration. This can be problematic in the case of antibody intermediates used to generate antibody drug conjugates (ADC) because the presence of non-ionic surfactants may interfere with the conjugation process. Therefore, there is a need for alternate surfactants that can protect against aggregation and particle formation.

Provided herein is an alternate surfactant that can protect against aggregation and particle formation in therapeutic biomolecule formulations. In particular, provided herein are stable pharmaceutical formulations that contain D-α-Tocopheryl polyethylene glycol succinate (TPGS) as a surfactant. As demonstrated herein, inclusion of TPGS can reduce particle formation in a therapeutic macromolecule formulation even as compared formulations that include non-ionic surfactants such as polysorbate 80, polysorbate 20 or poloxamer 188.

### D-α-Tocopheryl Polyethylene Glycol Succinate (TPGS)

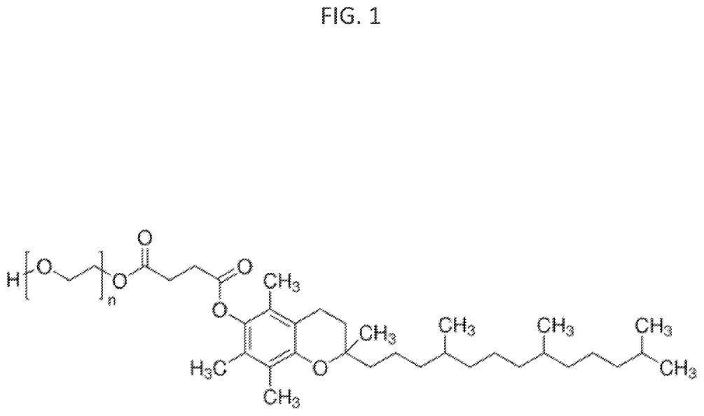

D-α-tocopheryl polyethylene glycol succinate (TPGS) (also referred to as tocophersolan) is a water-soluble synthetic derivative of natural α-tocopherol (vitamin E), formed by covalently joining tocopherol succinate, an ester formed through esterification of tocopherol and succinic acid, to a polyethylene glycol (PEG) moiety via an esterification reaction. A schematic of the structure for TPGS is shown in FIG. 1. TPGS has amphiphilic properties due to the presence of a polar hydrophilic head (polyethylene glycol) and a lipophilic tail (phytyl chain of d-α-tocopherol). TPGS has a melting point from about 37° C. to about 41° C., or about 38° C., is stable at a pH from about 4.5 to about 7.5 and has a solubility in water of about 20% at 20° C. Vitamin E TPGS is a highly stable form of Vitamin E. See, PMC Isochem. (2015). Vitamin E TPGS:NF and Food Grade. Available at pmcisochem.fr/page/info-center. TPGS is generally regarded as safe (GRAS) and has been approved by the Federal Drug Administration (FDA) as an inactive ingredient for oral and topical formulations.

TPGS compositions typically contain a mixture of monomers and dimers, where a monomer includes a single vitamin E molecule covalently joined to a water-soluble moiety, such as a polyethylene glycol (PEG), through a linker, in which the water-soluble moiety, e.g., PEG, has a free, unreacted, terminal reactive group, e.g., a free terminal hydroxyl group. A dimer includes two vitamin E molecules covalently joined to a water-soluble moiety, such as a polyethylene glycol (PEG), through one or more linkers, where both ends of the water-soluble moiety, e.g., both terminal hydroxyl groups of a PEG moiety, have reacted with a linker that is joined to a vitamin E molecule so that there are no free terminal reactive groups, e.g., hydroxyl groups. The monomers and dimers are formed during the esterification reaction. In one aspect, the TPGS compositions includes at least about 70%, about 75%, about 80%, about 85%, about 86%, about 87%, about 88%, about 89% or about 90% TPGS monomer and less than about 30%, about 25%, about 20%, about 15%, about 14%, about 13%, about 12%, about 11% or about 10% TPGS dimer. In one aspect, the TPGS composition includes at least about 85% TPGS monomer and less than about 15% TPGS dimer. The TPGS surfactant can include PEG moieties with a variety of molecular weights. In one aspect, the PEG moiety of TPGS has a molecular weight of about 1000 Da and the TPGS molecule is referred to D-α-tocopheryl polyethylene glycol 1000 succinate (TPGS 1000). As used herein, the term TPGS includes TPGS 1000.

Surfactants are typically amphiphilic molecules that contain both hydrophilic and lipophilic groups. The hydrophile-lipophile balance (HLB) number can be used as a measure of the ratio between these groups and can have a value between 0-60, which defines the affinity of a surfactant for water or oil. Molecules with a HLB number greater than 10 have an affinity for water (hydrophilic) and molecules with a HLB number less than 10 have an affinity for oil (lipophilic). Non-ionic surfactants have HLB numbers ranging from 0-20. TPGS 1000 has a HLB number of 13.2. See, Wu and Hopkins. (1999). Characteristics of D-alpha-tocopheryl PEG1000 succinate for applications as an absorption enhancer in drug delivery systems. Pharm Tech. 23:52-60. The critical micelle concentration (CMC) is the concentration at and above which the surfactant forms micelles. Below the CMC, the surface tension decreases with increasing surfactant concentration. Above the CMC, additional surfactant added to the system forms micelles. TPGS 1000 has a CMC of 0.02% (w/w) at 37% C. See, Wu and Hopkins. (1999). Characteristics of D-alpha-tocopheryl PEG1000 succinate for applications as an absorption enhancer in drug delivery systems. Pharm Tech. 23:52-60.

### Stable Pharmaceutical Formulation

In one aspect, a stable pharmaceutical formulation is provided. In one aspect, the pharmaceutical formulation has an improved stability against stress, including, for example, mechanical stress, thermal stress or stress resulting from freezing and thawing. In one aspect, the bioactive agent in the stable pharmaceutical formulation retains its physical stability, chemical stability or biological activity during storage.

In one aspect, the pharmaceutical formulation includes D-α-Tocopheryl polyethylene glycol succinate (TPGS). In one aspect, the pharmaceutical formulation includes D-α-Tocopheryl polyethylene glycol 1000 succinate (TPGS 1000). In one aspect, the pharmaceutical formulation includes D-α-Tocopheryl polyethylene glycol succinate (TPGS) as a surfactant. In one aspect, the pharmaceutical formulation includes D-α-Tocopheryl polyethylene glycol 1000 succinate (TPGS 1000) as a surfactant. Surfactants can be included in a pharmaceutical formulation for a variety of reasons including, for example, to prevent or control aggregation, particle formation or surface adsorption in liquid formulations or to prevent or control these phenomena during lyophilization or reconstitution of lyophilized formulations. In one aspect, the stable pharmaceutical formulation does not include a non-ionic surfactant. In one aspect, the stable pharmaceutical formulation does not include a poly-oxy-ethylene (PEO) based surfactant such as polysorbate 80, polysorbate 20, poloxamer 188, or combinations thereof.

In one aspect, the physical stability, chemical stability or biological activity of the bioactive agent in the pharmaceutical formulation at a given time is within about 0.1%, about 0.5%, about 1%, about 5%, about 10%, about 15%, about 20% or about 25% (within a standard of error) of the physical stability, chemical stability or biological activity of the bioactive agent exhibited at an initial time point, for example, at the time the pharmaceutical formulation was prepared (TO).

Generally, stability is determined with respect to a selected temperature and a selected time-period. In one aspect, the formulation is stable at a temperature of about 30° C.±10° C., about ±5° C. or about 30° C.±2° C., or at about 30° C., about 35° C. or about 40° C. for at least about 2 weeks, about 1 month, about 2 months or about 3 months and up to about 6 months. In one aspect, the formulation is stable at a temperature of about 20° C.±10° C., about 20° C.±5° C. or about 20° C.±2° C., or at about 15° C., about 20° C., about 25° C. or about 30° C. for at least about 1 month, about 2 months or about 3 months and up to about 6 months, about 9 months or about 12 months. In one aspect, the formulation is stable at a temperature from about 5° C.±5° C. or about ±3° C., or between about 2° C. to about 8° C., or at about 2° C., about 4° C., about 6° C. or about 8° C. for at least about 1 month, about 3 months, or about 6 months and up to about 9 months, about 12 months, about 24 months or about 36 months. In one aspect, the formulation is stable at a temperature from about −20° C.±10° C. or about −20° C.±5° C., or at about −25° C., about −20° C., about −15° C. or about −10° C. for at least about 1 month, about 3 months, or about 6 months and up to about 9 months, about 12 months, about 24 months or about 36 months. In one aspect, the formulation is stable at a temperature from about −70° C.±10° C., or about −70° C.±5° C. or at about −70° C., about −75° C. or about −80° C. for at least about 1 month, about 3 months, or about 6 months and up to about 9 months, about 12 months, about 24 months or about 36 months.

In one aspect, the formulation is stable at 40° C. for up to about 3 months. In one aspect, the formulation is stable at 40° C. for up to about 6 months. In one aspect, the formulation is stable at 25° C. for up to about 6 months. In one aspect, the formulation is stable at 25° C. for up to about 12 months. In one aspect, the formulation is stable at a temperature from about 2° C. to about 8° C. for up to about 6 months. In one aspect, the formulation is stable at a temperature from about 2° C. to about 8° C. for up to about 12 months. In one aspect, the formulation is stable at a temperature from about 2° C. to about 8° C. for up to about 24 months. In one aspect, the formulation is stable at a temperature from about 2° C. to about 8° C. for up to about 36 months. In one aspect, the formulation is stable at −20° C. for up to about 6 months. In one aspect, the formulation is stable at −20° C. for up to about 12 months. In one aspect, the formulation is stable at −20° C. for up to about 24 months. In one aspect, the formulation is stable at −20° C. for up to about 36 months. In one aspect, the formulation is stable at −80° C. for up to about 6 months. In one aspect, the formulation is stable at −80° C. for up to about 12 months. In one aspect, the formulation is stable at −80° C. for up to about 24 months. In one aspect, the formulation is stable at −80° C. for up to about 36 months.

In one aspect, the bioactive agent in the pharmaceutical formulation is chemically stable. In one aspect, there is an increase of less than about 25%, about 20%, about 15%, about 10% or about 5% in chemically altered forms of the bioactive agent in the pharmaceutical formulation when stored for at least about 2 weeks, about 1 month, about 2 months, about 3 months or about 6 months and up to about 9 months, about 12 months, about 24 months or about 36 months at a temperature of about 40° C., about 25° C., about 2° C. to about 8° C., about −20° C. or about −80° C. In one aspect, there is less than about 25%, about 20%, about 15%, about 10% or about 5% increase in one or more chemically altered forms of the bioactive agent, including, for example, chemically altered forms due to deamidation, including, for example, asparagine (Asn) deamidation; oxidation, including for example methionine, cysteine, histidine, tyrosine, tryptophan or phenylalanine oxidation; intra- and inter-residue cyclization (aspartic and glutamic acid, asparagine, glutamine, N-terminal dipeptidyl motifs); clipping/hydrolysis/fragmentation; (β-elimination; glycation; and disulfide scrambling.

In one aspect, the bioactive agent in the pharmaceutical formulation is physically stable. In one aspect, there is an increase of less than about 25%, about 20%, about 15%, about 10% or about 5% in physically altered forms of the bioactive agent in the pharmaceutical formulation when stored for at least about 2 weeks, about 1 month, about 2 months, about 3 months or about 6 months and up to about 9 months, about 12 months, about 24 months or about 36 months at a temperature of about 40° C., about 25° C., about 2° C. to about 8° C., about −20° C. or about −80° C. In one aspect, there is less than about 25%, about 20%, about 15%, about 10% or about 5% increase in one or more physically altered forms of the bioactive agent, including, for example, physically altered forms due to denaturation, aggregation, precipitation, or particulate formation.

In one aspect, the bioactive agent in the biopharmaceutical formulation is biologically stable. In one aspect, there is a decrease of less than about 25%, about 20%, about 15%, about 10% or about 5% in a biological activity of the bioactive agent in the pharmaceutical formulation when stored for at least about 2 weeks, about 1 month, about 2 months, about 3 months or about 6 months and up to about 9 months, about 12 months, about 24 months or about 36 months at a temperature of about 40° C., about 25° C., about 2° C. to about 8° C., about −20° C. or about −80° C. In one aspect, there is a decrease of less than about 25%, about 20%, about 15%, about 10% or about 5% in a biological activity including, but not limited to, enzymatic activity; ability to interact with or bind to another molecule; ability to activate, promote, stabilize, inhibit, suppress, or destabilize a function of another molecule; ability to produce a specific protein; ability to impart resistance; and ability to localize at a specific position in a cell.

In one aspect, the stability of a pharmaceutical formulation is evaluated by measuring the amount of particle impurities. Particle impurities can include visible, subvisible and submicron impurities. Visible impurities have a diameter of greater than about 100 μm or about 150 μm and can be detected by visual inspection. Sub-visible particles generally range in size from about 1 μm to about 100 μm or about 150 μm. Submicron particles have a diameter of less than about 1 μm. Sub-visible particles generally pose the greatest risk when present in pharmaceutical formulations, particularly when present in pharmaceutical formulations for parenteral administration, including subcutaneous, intravenous or intramuscular administration, due, in some cases, to the ability of sub-visible particles to elicit an adverse immunogenic response. Sub-visible particles with a diameter of greater than or equal to about 10 μm or greater than or equal to about 5 μm can block blood vessels in the lungs following vascular infusion. Methods for detecting and quantifying particulate impurities are known and include Dynamic Light Scattering (DLS) or Static Light Scattering (SLS), Nanoparticle Tracking Analysis (NTA), light microscopy, Electrical Sensing Zone (ESZ), Flow-Imaging Technology, Resonant Mass measurement, Electron Microscopy, Fourier Transform Infrared (FTIR) Microscopy, and Raman Microscopy. In one aspect, sub-visible particles are detected using Micro Flow Imaging (MFI).

In one aspect, a stable pharmaceutical formulation is provided that includes less than about 10,000, about 5,000, about 1,000, about 750, about 500, about 250, about 150, about 100, or about 50 particles greater than about 2 μm, about 5 μm, about 10 μm, about 15 μm, about 20 μm or about 25 μm diameter/mL when stored for at least about 2 weeks, about 1 month, about 2 months, about 3 months or about 6 months and up to about 9 months, about 12 months, about 24 months or about 36 months at a temperature of about 30° C.±10° C., about 30° C.±5° C., about 20° C.±10° C., about 20° C.±5° C., about 20° C.±2° C., about 5° C.±5° C., about 5° C.±3° C., about −20° C. about −20° C.±5° C., about −70° C.±10° C., or about −70° C.±5° C.

In one aspect, a stable pharmaceutical formulation is provided that includes less than about 5,000 particles greater than about 2 μm diameter/mL when stored for at least about 2 weeks, about 1 month, about 2 months, about 3 months or about 6 months and up to about 9 months, about 12 months, about 24 months or about 36 months at a temperature of about 30° C.±10° C., about 20° C.±10° C., about 5° C.±3° C., about −20° C.±10° C. or about −70° C.±10° C. In one aspect, the pharmaceutical formulation includes less than about 5,000 particles greater than about 2 μm in diameter/mL when stored at a temperature of about 30° C.±10° C. for up to about 3 months. In one aspect, the pharmaceutical formulation includes less than about 5,000 particles greater than about 2 μm in diameter/mL when stored at a temperature of about 30° C.±10° C. for up to about 6 months. In one aspect, the pharmaceutical formulation includes less than about particles greater than about 2 μm in diameter/mL when stored at a temperature of about ±10° C. for up to about 6 months. In one aspect, the pharmaceutical formulation includes less than about 5,000 particles greater than about 2 μm in diameter/mL when stored at a temperature of about 20° C.±5° C. for up to about 12 months. In one aspect, the pharmaceutical formulation includes less than about 5,000 particles greater than about 2 μm in diameter/mL when stored at a temperature of about 5° C.±3° C. for up to about 12 months. In one aspect, the pharmaceutical formulation includes less than about 5,000 particles greater than about 2 μm in diameter/mL when stored at a temperature of about 5° C.±3° C. for up to about 24 months. In one aspect, the pharmaceutical formulation includes less than about 5,000 particles greater than about 2 μm in diameter/mL when stored at a temperature of about 5° C.±3° C. for up to about 36 months. In one aspect, the pharmaceutical formulation includes less than about 5,000 particles greater than about 2 μm in diameter/mL when stored at a temperature of about −20° C.±10° C. for up to about 6 months. In one aspect, the pharmaceutical formulation includes less than about particles greater than about 2 μm in diameter/mL when stored at a temperature of about −±10° C. for up to about 12 months. In one aspect, the pharmaceutical formulation includes less than about 5,000 particles greater than about 2 μm in diameter/mL when stored at a temperature of about −20° C.±10° C. for up to about 24 months. In one aspect, the pharmaceutical formulation includes less than about 5,000 particles greater than about 2 μm in diameter/mL when stored at a temperature of about −20° C.±10° C. for up to about 36 months. In one aspect, the pharmaceutical formulation includes less than about 5,000 particles greater than about 2 μm in diameter/mL when stored at a temperature of about −70° C.±10° C. for up to about 6 months. In one aspect, the pharmaceutical formulation includes less than about 5,000 particles greater than about 2 μm in diameter/mL when stored at a temperature of about −70° C.±10° C. for up to about 12 months. In one aspect, the pharmaceutical formulation includes less than about 5,000 particles greater than about 2 μm in diameter/mL when stored at a temperature of about −70° C.±10° C. for up to about 24 months. In one aspect, the pharmaceutical formulation includes less than about particles greater than about 2 μm in diameter/mL when stored at a temperature of about −70° C.±10° C. for up to about 36 months.

In one aspect, the pharmaceutical formulation includes less than about 5,000 particles greater than about 2 μm in diameter/mL when stored at a temperature of about 40° C. for up to about 3 months. In one aspect, the pharmaceutical formulation includes less than about 5,000 particles greater than about 2 μm in diameter/mL when stored at a temperature of about 40° C. for up to 6 months. In one aspect, the pharmaceutical formulation includes less than about 5,000 particles greater than about 2 μm in diameter/mL when stored at a temperature of about 25° C. for up to about 6 months. In one aspect, the pharmaceutical formulation includes less than about 5,000 particles greater than about 2 μm in diameter/mL when stored at a temperature of about 25° C. for up to about 12 months. In one aspect, the pharmaceutical formulation includes less than about 5,000 particles greater than about 2 μm in diameter/mL when stored at a temperature of about 2° C. to about 8° C. for up to about 12 months. In one aspect, the pharmaceutical formulation includes less than about 5,000 particles greater than about 2 μm in diameter/mL when stored at a temperature of about 2° C. to about 8° C. for up to about 24 months. In one aspect, the pharmaceutical formulation includes less than about 5,000 particles greater than about 2 μm in diameter/mL when stored at a temperature of about 2° C. to about 8° C. for up to about 36 months. In one aspect, the pharmaceutical formulation includes less than about 5,000 particles greater than about 2 μm in diameter/mL when stored at a temperature of about −20° C. for up to about 6 months. In one aspect, the pharmaceutical formulation includes less than about 5,000 particles greater than about 2 μm in diameter/mL when stored at a temperature of about −20° C. for up to about 12 months. In one aspect, the pharmaceutical formulation includes less than about 5,000 particles greater than about 2 μm in diameter/mL when stored at a temperature of about −20° C. for up to about 24 months. In one aspect, the pharmaceutical formulation includes less than about 5,000 particles greater than about 2 μm in diameter/mL when stored at a temperature of about −20° C. for up to about 36 months. In one aspect, the pharmaceutical formulation includes less than about 5,000 particles greater than about 2 μm in diameter/mL when stored at a temperature of about −80° C. for up to about 6 months. In one aspect, the pharmaceutical formulation includes less than about 5,000 particles greater than about 2 μm in diameter/mL when stored at a temperature of about −80° C. for up to about 12 months. In one aspect, the pharmaceutical formulation includes less than about 5,000 particles greater than about 2 μm in diameter/mL when stored at a temperature of about −80° C. for up to about 24 months. In one aspect, the pharmaceutical formulation includes less than about 5,000 particles greater than about 2 μm in diameter/mL when stored at a temperature of about −80° C. for up to about 36 months.

In one aspect, a stable pharmaceutical formulation is provided that includes less than about 1,000 particles greater than about 2 μm diameter/mL when stored for at least about 2 weeks, about 1 month, about 2 months, about 3 months or about 6 months and up to about 9 months, about 12 months, about 24 months or about 36 months at a temperature of about 30° C.±10° C., about 20° C.±10° C., about 5° C.±3° C., about −20° C.±10° C. or about −70° C.±10° C. In one aspect, the pharmaceutical formulation includes less than about 1,000 particles greater than about 2 μm in diameter/mL when stored at a temperature of about 30° C.±10° C. for up to about 3 months. In one aspect, the pharmaceutical formulation includes less than about 1,000 particles greater than about 2 μm in diameter/mL when stored at a temperature of about 30° C.±10° C. for up to about 6 months. In one aspect, the pharmaceutical formulation includes less than about 1,000 particles greater than about 2 μm in diameter/mL when stored at a temperature of about ±10° C. for up to about 6 months. In one aspect, the pharmaceutical formulation includes less than about 1,000 particles greater than about 2 μm in diameter/mL when stored at a temperature of about 20° C.±5° C. for up to about 12 months. In one aspect, the pharmaceutical formulation includes less than about 1,000 particles greater than about 2 μm in diameter/mL when stored at a temperature of about 5° C.±3° C. for up to about 12 months. In one aspect, the pharmaceutical formulation includes less than about 1,000 particles greater than about 2 μm in diameter/mL when stored at a temperature of about 5° C.±3° C. for up to about 24 months. In one aspect, the pharmaceutical formulation includes less than about 1,000 particles greater than about 2 μm in diameter/mL when stored at a temperature of about 5° C.±3° C. for up to about 36 months. In one aspect, the pharmaceutical formulation includes less than about 1,000 particles greater than about 2 μm in diameter/mL when stored at a temperature of about −20° C.±10° C. for up to about 6 months. In one aspect, the pharmaceutical formulation includes less than about 1,000 particles greater than about 2 μm in diameter/mL when stored at a temperature of about −±10° C. for up to about 12 months. In one aspect, the pharmaceutical formulation includes less than about 1,000 particles greater than about 2 μm in diameter/mL when stored at a temperature of about −20° C.±10° C. for up to about 24 months. In one aspect, the pharmaceutical formulation includes less than about 1,000 particles greater than about 2 μm in diameter/mL when stored at a temperature of about −20° C.±10° C. for up to about 36 months. In one aspect, the pharmaceutical formulation includes less than about 1,000 particles greater than about 2 μm in diameter/mL when stored at a temperature of about −70° C.±10° C. for up to about 6 months. In one aspect, the pharmaceutical formulation includes less than about 1,000 particles greater than about 2 μm in diameter/mL when stored at a temperature of about −70° C.±10° C. for up to about 12 months. In one aspect, the pharmaceutical formulation includes less than about 1,000 particles greater than about 2 μm in diameter/mL when stored at a temperature of about −70° C.±10° C. for up to about 24 months. In one aspect, the pharmaceutical formulation includes less than about 1,000 particles greater than about 2 μm in diameter/mL when stored at a temperature of about −70° C.±10° C. for up to about 36 months.

In one aspect, the pharmaceutical formulation includes less than about 1,000 particles greater than about 2 μm in diameter/mL when stored at a temperature of about 40° C. for up to about 3 months. In one aspect, the pharmaceutical formulation includes less than about 1,000 particles greater than about 2 μm in diameter/mL when stored at a temperature of about 40° C. for up to about 6 months. In one aspect, the pharmaceutical formulation includes less than about 1,000 particles greater than about 2 μm in diameter/mL when stored at a temperature of about for up to about 6 months. In one aspect, the pharmaceutical formulation includes less than about 1,000 particles greater than about 2 μm in diameter/mL when stored at a temperature of about 25° C. for up to about 12 months. In one aspect, the pharmaceutical formulation includes less than about 1,000 particles greater than about 2 μm in diameter/mL when stored at a temperature of about 2° C. to about 8° C. for up to about 12 months. In one aspect, the pharmaceutical formulation includes less than about 1,000 particles greater than about 2 μm in diameter/mL when stored at a temperature of about 2° C. to about 8° C. for up to about 24 months. In one aspect, the pharmaceutical formulation includes less than about 1,000 particles greater than about 2 μm in diameter/mL when stored at a temperature of about 2° C. to about 8° C. for up to about 36 months. In one aspect, the pharmaceutical formulation includes less than about 1,000 particles greater than about 2 μm in diameter/mL when stored at a temperature of about −20° C. for up to about 6 months. In one aspect, the pharmaceutical formulation includes less than about 1,000 particles greater than about 2 μm in diameter/mL when stored at a temperature of about −20° C. for up to about 12 months. In one aspect, the pharmaceutical formulation includes less than about 1,000 particles greater than about 2 μm in diameter/mL when stored at a temperature of about −20° C. for up to about 24 months. In one aspect, the pharmaceutical formulation includes less than about 1,000 particles greater than about 2 μm in diameter/mL when stored at a temperature of about −20° C. for up to about 36 months. In one aspect, the pharmaceutical formulation includes less than about 1,000 particles greater than about 2 μm in diameter/mL when stored at a temperature of about −80° C. for up to about 6 months. In one aspect, the pharmaceutical formulation includes less than about 1,000 particles greater than about 2 μm in diameter/mL when stored at a temperature of about −80° C. for up to about 12 months. In one aspect, the pharmaceutical formulation includes less than about 1,000 particles greater than about 2 μm in diameter/mL when stored at a temperature of about −80° C. for up to about 24 months. In one aspect, the pharmaceutical formulation includes less than about 1,000 particles greater than about 2 μm in diameter/mL when stored at a temperature of about −80° C. for up to about 36 months.

In one aspect, a stable pharmaceutical formulation is provided that includes less than about 6,000 particles greater than about 10 μm diameter/mL when stored for at least about 2 weeks, about 1 month, about 2 months, about 3 months or about 6 months and up to about 9 months, about 12 months or about 24 months at a temperature of about 30° C.±10° C., about 20° C.±10° C., about 5° C.±3° C., about −20° C.±10° C. or about −70° C.±10° C. In one aspect, the pharmaceutical formulation includes less than about 6,000 particles greater than about 10 μm in diameter/mL when stored at a temperature of about 30° C.±10° C. for up to about 3 months. In one aspect, the pharmaceutical formulation includes less than about 6,000 particles greater than about μm in diameter/mL when stored at a temperature of about 30° C.±10° C. for up to about 6 months. In one aspect, the pharmaceutical formulation includes less than about 6,000 particles greater than about 10 μm in diameter/mL when stored at a temperature of about 20° C.±10° C. for up to about 6 months. In one aspect, the pharmaceutical formulation includes less than about 6,000 particles greater than about 10 μm in diameter/mL when stored at a temperature of about ±5° C. for up to about 12 months. In one aspect, the pharmaceutical formulation includes less than about 6,000 particles greater than about 10 μm in diameter/mL when stored at a temperature of about 5° C.±3° C. for up to about 12 months. In one aspect, the pharmaceutical formulation includes less than about 6,000 particles greater than about 10 μm in diameter/mL when stored at a temperature of about 5° C.±3° C. for up to about 24 months. In one aspect, the pharmaceutical formulation includes less than about 6,000 particles greater than about 10 μm in diameter/mL when stored at a temperature of about 5° C.±3° C. for up to about 36 months. In one aspect, the pharmaceutical formulation includes less than about 6,000 particles greater than about 10 μm in diameter/mL when stored at a temperature of about −20° C.±10° C. for up to about 6 months. In one aspect, the pharmaceutical formulation includes less than about 6,000 particles greater than about 10 μm in diameter/mL when stored at a temperature of about −20° C.±10° C. for up to about 12 months. In one aspect, the pharmaceutical formulation includes less than about 6,000 particles greater than about 10 μm in diameter/mL when stored at a temperature of about −20° C.±10° C. for up to about 24 months. In one aspect, the pharmaceutical formulation includes less than about 6,000 particles greater than about 10 μm in diameter/mL when stored at a temperature of about −20° C.±10° C. for up to about 36 months. In one aspect, the pharmaceutical formulation includes less than about 6,000 particles greater than about 10 μm in diameter/mL when stored at a temperature of about −70° C.±10° C. for up to about 6 months. In one aspect, the pharmaceutical formulation includes less than about 6,000 particles greater than about 10 μm in diameter/mL when stored at a temperature of about −70° C.±10° C. for up to about 12 months. In one aspect, the pharmaceutical formulation includes less than about 6,000 particles greater than about 10 μm in diameter/mL when stored at a temperature of about −70° C.±10° C. for up to about 24 months. In one aspect, the pharmaceutical formulation includes less than about 6,000 particles greater than about 10 μm in diameter/mL when stored at a temperature of about −70° C.±10° C. for up to about 36 months.

In one aspect, the pharmaceutical formulation includes less than about 6,000 particles greater than about 10 μm in diameter/mL when stored at a temperature of about 40° C. for up to about 3 months. In one aspect, the pharmaceutical formulation includes less than about 6,000 particles greater than about 10 μm in diameter/mL when stored at a temperature of about 40° C. for up to about 6 months. In one aspect, the pharmaceutical formulation includes less than about 6,000 particles greater than about 10 μm in diameter/mL when stored at a temperature of about for up to about 6 months. In one aspect, the pharmaceutical formulation includes less than about 6,000 particles greater than about 10 μm in diameter/mL when stored at a temperature of about 25° C. for up to about 12 months. In one aspect, the pharmaceutical formulation includes less than about 6,000 particles greater than about 10 μm in diameter/mL when stored at a temperature of about 2° C. to about 8° C. for up to about 12 months. In one aspect, the pharmaceutical formulation includes less than about 6,000 particles greater than about 10 μm in diameter/mL when stored at a temperature of about 2° C. to about 8° C. for up to about 24 months. In one aspect, the pharmaceutical formulation includes less than about 6,000 particles greater than about 10 μm in diameter/mL when stored at a temperature of about 2° C. to about 8° C. for up to about 36 months. In one aspect, the pharmaceutical formulation includes less than about 6,000 particles greater than about 10 μm in diameter/mL when stored at a temperature of about −20° C. for up to about 6 months. In one aspect, the pharmaceutical formulation includes less than about 6,000 particles greater than about 10 μm in diameter/mL when stored at a temperature of about −20° C. for up to about 12 months. In one aspect, the pharmaceutical formulation includes less than about 6,000 particles greater than about 10 μm in diameter/mL when stored at a temperature of about −20° C. for up to about 24 months. In one aspect, the pharmaceutical formulation includes less than about 6,000 particles greater than about 10 μm in diameter/mL when stored at a temperature of about −20° C. for up to about 36 months. In one aspect, the pharmaceutical formulation includes less than about 6,000 particles greater than about 10 μm in diameter/mL when stored at a temperature of about −80° C. for up to about 6 months. In one aspect, the pharmaceutical formulation includes less than about 6,000 particles greater than about 10 μm in diameter/mL when stored at a temperature of about −80° C. for up to about 12 months. In one aspect, the pharmaceutical formulation includes less than about 6,000 particles greater than about μm in diameter/mL when stored at a temperature of about −80° C. for up to about 24 months. In one aspect, the pharmaceutical formulation includes less than about 6,000 particles greater than about 10 μm in diameter/mL when stored at a temperature of about −80° C. for up to about 36 months.

In one aspect, a stable pharmaceutical formulation is provided that includes less than about 600 particles greater than about 25 μm diameter/mL when stored for at least about 2 weeks, about 1 month, about 2 months, about 3 months or about 6 months and up to about 9 months, about 12 months or about 24 months at a temperature of about 30° C.±10° C., about 20° C.±10° C., about 5° C.±3° C., about −20° C.±10° C. or about −70° C.±10° C. In one aspect, the pharmaceutical formulation includes less than about 600 particles greater than about 25 μm in diameter/mL when stored at a temperature of about 30° C.±10° C. for up to about 3 months. In one aspect, the pharmaceutical formulation includes less than about 600 particles greater than about μm in diameter/mL when stored at a temperature of about 30° C.±10° C. for up to about 6 months. In one aspect, the pharmaceutical formulation includes less than about 600 particles greater than about 25 μm in diameter/mL when stored at a temperature of about 20° C.±10° C. for up to about 6 months. In one aspect, the pharmaceutical formulation includes less than about 600 particles greater than about 25 μm in diameter/mL when stored at a temperature of about 20° C.±5° C. for up to about 12 months. In one aspect, the pharmaceutical formulation includes less than about 600 particles greater than about 25 μm in diameter/mL when stored at a temperature of about 5° C.±3° C. for up to about 12 months. In one aspect, the pharmaceutical formulation includes less than about 600 particles greater than about 25 μm in diameter/mL when stored at a temperature of about 5° C.±3° C. for up to about 24 months. In one aspect, the pharmaceutical formulation includes less than about 600 particles greater than about 25 μm in diameter/mL when stored at a temperature of about 5° C.±3° C. for up to about 36 months. In one aspect, the pharmaceutical formulation includes less than about 600 particles greater than about 25 μm in diameter/mL when stored at a temperature of about −20° C.±10° C. for up to about 6 months. In one aspect, the pharmaceutical formulation includes less than about 600 particles greater than about 25 μm in diameter/mL when stored at a temperature of about −20° C.±10° C. for up to about 12 months. In one aspect, the pharmaceutical formulation includes less than about 600 particles greater than about 25 μm in diameter/mL when stored at a temperature of about −20° C.±10° C. for up to about 24 months. In one aspect, the pharmaceutical formulation includes less than about 600 particles greater than about 25 μm in diameter/mL when stored at a temperature of about −±10° C. for up to about 36 months. In one aspect, the pharmaceutical formulation includes less than about 600 particles greater than about 25 μm in diameter/mL when stored at a temperature of about −70° C.±10° C. for up to about 6 months. In one aspect, the pharmaceutical formulation includes less than about 600 particles greater than about 25 μm in diameter/mL when stored at a temperature of about −70° C.±10° C. for up to about 12 months. In one aspect, the pharmaceutical formulation includes less than about 600 particles greater than about 25 μm in diameter/mL when stored at a temperature of about −70° C.±10° C. for up to about 24 months. In one aspect, the pharmaceutical formulation includes less than about 600 particles greater than about 25 μm in diameter/mL when stored at a temperature of about −70° C.±10° C. for up to about 36 months.

In one aspect, the pharmaceutical formulation includes less than about 600 particles greater than about 25 μm in diameter/mL when stored at a temperature of about 40° C. for up to about 3 months. In one aspect, the pharmaceutical formulation includes less than about 600 particles greater than about 25 μm in diameter/mL when stored at a temperature of about 40° C. for up to about 6 months. In one aspect, the pharmaceutical formulation includes less than about 600 particles greater than about 25 μm in diameter/mL when stored at a temperature of about for up to about 6 months. In one aspect, the pharmaceutical formulation includes less than about 600 particles greater than about 25 μm in diameter/mL when stored at a temperature of about 25° C. for up to about 12 months. In one aspect, the pharmaceutical formulation includes less than about 600 particles greater than about 25 μm in diameter/mL when stored at a temperature of about 2° C. to about 8° C. for up to about 12 months. In one aspect, the pharmaceutical formulation includes less than about 600 particles greater than about 25 μm in diameter/mL when stored at a temperature of about 2° C. to about 8° C. for up to about 24 months. In one aspect, the pharmaceutical formulation includes less than about 600 particles greater than about 25 μm in diameter/mL when stored at a temperature of about 2° C. to about 8° C. for up to about 36 months. In one aspect, the pharmaceutical formulation includes less than about 600 particles greater than about 25 μm in diameter/mL when stored at a temperature of about −20° C. for up to about 6 months. In one aspect, the pharmaceutical formulation includes less than about 600 particles greater than about 25 μm in diameter/mL when stored at a temperature of about −20° C. for up to about 12 months. In one aspect, the pharmaceutical formulation includes less than about 600 particles greater than about 25 μm in diameter/mL when stored at a temperature of about −20° C. for up to about 24 months. In one aspect, the pharmaceutical formulation includes less than about 600 particles greater than about 25 μm in diameter/mL when stored at a temperature of about −20° C. for up to about 36 months. In one aspect, the pharmaceutical formulation includes less than about 600 particles greater than about 25 μm in diameter/mL when stored at a temperature of about −80° C. for up to about 6 months. In one aspect, the pharmaceutical formulation includes less than about 600 particles greater than about 25 μm in diameter/mL when stored at a temperature of about −80° C. for up to about 12 months. In one aspect, the pharmaceutical formulation includes less than about 600 particles greater than about μm in diameter/mL when stored at a temperature of about −80° C. for up to about 24 months. In one aspect, the pharmaceutical formulation includes less than about 600 particles greater than about 25 μm in diameter/mL when stored at a temperature of about −80° C. for up to about 36 months.

In one aspect, a method of reducing particle formation in an aqueous pharmaceutical formulation is provided. In one aspect, the method includes adding D-α-Tocopheryl polyethylene glycol succinate (TPGS) to the formulation. In one aspect, the method includes adding D-α-Tocopheryl polyethylene glycol 1000 succinate (TPGS 1000) to the formulation. In one aspect, less than about 10,000, about 5,000, about 1,000, about 750, about 500, about 250, about 150, about 100, or about 50 particles greater than about 2 μm, about 5 μm, about 10 μm, about 15 μm, about 20 μm or about 25 μm diameter/mL are formed after at least about 2 weeks, about 1 month, about 2 months, about 3 months or about 6 months and up to about 9 months, about 12 months, about 24 months or about 36 months at a temperature of about 30° C.±10° C., about 30° C.±5° C., about 20° C.±10° C., about 20° C.±5° C., about 20° C.±2° C., about 5° C.±5° C., about 5° C.±3° C., about −20° C.±10° C., about −20° C.±5° C., about −70° C.±10° C., or about −70° C.±5° C. In one aspect, the pharmaceutical formulation includes less than about 5,000 particles greater than about 2 μm in diameter/mL. In one aspect, the pharmaceutical formulation includes less than about 1,000 particles greater than about 2 μm in diameter/mL. In one aspect, the pharmaceutical formulation includes less than about 6,000 particles greater than about 10 μm in diameter/mL. In one aspect, the pharmaceutical formulation includes less than about 600 particles greater than about 25 μm in diameter/mL.

In one aspect, the pharmaceutical formulation includes TPGS at a concentration from about 0.001% to about 1%, or from about 0.01% to about 1% or from about 0.01% to about 0.1%. In one aspect, the pharmaceutical formulation includes TPGS at a concentration from about 0.001%, about 0.005%, about 0.01%, about 0.02%, about 0.03%, about 0.04%, or about and up to about 0.1%, about 0.2%, about 0.3%, about 0.4%, about 0.5%, or about 1%. In one aspect, the pharmaceutical formulation includes TPGS in an amount of about 0.01%, about about 0.03%, about 0.04%, or about 0.05%. In one aspect, the pharmaceutical formulation includes TPGS in an amount of about 0.01%. In one aspect, the pharmaceutical formulation includes TPGS in an amount of about 0.02%. In one aspect, the pharmaceutical formulation includes TPGS in an amount of about 0.03%. In one aspect, the pharmaceutical formulation includes TPGS in an amount of about 0.04%. In one aspect, the pharmaceutical formulation includes TPGS in an amount of about 0.05%.

In one aspect, the pharmaceutical formulation includes TPGS 1000 at a concentration from about 0.001% to about 1%, or from about 0.01% to about 1% or from about 0.01% to about 0.1%. In one aspect, the pharmaceutical formulation includes TPGS 1000 at a concentration from about about 0.005%, about 0.01%, about 0.02%, about 0.03%, about 0.04%, or about 0.05% and up to about 0.1%, about 0.2%, about 0.3%, about 0.4%, about 0.5%, or about 1%. In one aspect, the pharmaceutical formulation includes TPGS 1000 in an amount of about 0.01%, about about 0.03%, about 0.04%, or about 0.05%. In one aspect, the pharmaceutical formulation includes TPGS 1000 in an amount of about 0.01%. In one aspect, the pharmaceutical formulation includes TPGS 1000 in an amount of about 0.02%. In one aspect, the pharmaceutical formulation includes TPGS 1000 in an amount of about 0.03%. In one aspect, the pharmaceutical formulation includes TPGS 1000 in an amount of about 0.04%. In one aspect, the pharmaceutical formulation includes TPGS 1000 in an amount of about 0.05%.

In one aspect, the pharmaceutical formulation is lyophilized. “Lyophilization” refers to a dehydration process in which a liquid formulation is frozen and the surrounding pressure is reduced to allow for sublimation of the frozen solvent. Lyophilization methods are known in the art. Lyophilization results in a cake or powder which can be stored over a long time-period. Prior to administration, the lyophilized composition is reconstituted using a solvent, for example, sterile water for injection. A “reconstituted formulation” refers to a lyophilized composition after solubilization.

In one aspect the pharmaceutical formulation is a liquid formulation. In one aspect, a “liquid formulation” includes reconstituted lyophilized compositions. In one aspect, the liquid formulation is not lyophilized and has not been subjected to lyophilization. In one aspect, the liquid formulation is an aqueous formulation, in which the solvent includes water.

In one aspect, the pharmaceutical formulation includes a bioactive agent at a concentration from about 0.1 mg/mL to about 300 mg/mL. In one aspect, the pharmaceutical formulation includes an antibody or antigen-binding antibody fragment as a bioactive agent at a concentration from about 10 mg/mL to about 200 mg/mL. In one aspect, the concentration of antibody or antigen-binding antibody fragment is at least about 10 mg/mL, about 15 mg/mL, about 20 mg/mL, about mg/mL, about 50 mg/mL, about 75 mg/mL or about 100 mg/mL and up to about 125 mg/mL, about 150 mg/mL, about 175 mg/mL, about 200 mg/mL, about 250 mg/mL or about 300 mg/mL.

In one aspect, the pharmaceutical formulation includes a therapeutic protein as a bioactive agent at a concentration from about 0.1 mg/mL to about 100 mg/mL, or about 1 mg/mL to about mg/mL. In one aspect, the pharmaceutical formulation includes a therapeutic protein as a bioactive agent at a concentration from about 0.1 mg/mL, about 0.5 mg/mL, about 1 mg/mL, about 2 mg/mL, about 3 mg/mL, about 4 mg/mL or about 5 mg/mL and up to about 10 mg/mL, about 15 mg/mL, about 20 mg/mL, about 25 mgL, about 30 mg/mL, about 35 mg/mL, about 35 mg/mL, about 40 mg/mL, about 45 mg/mL, about 50 mg/mL, about 75 mg/mL or about 100 mg/mL.

In one aspect, the formulation includes a buffer at a concentration from about 0.1 mM to about 100 mM, about 0.5 mM to about 50 mM, about 10 mM to about 50 mM, or about 10 mM to about 30 mM. In one aspect, the formulation includes a buffer at a concentration from about 0.1 mM, about 0.5 mM, about 1 mM, about 5 mM, about 10 mM, about 20 mM or about 25 mM and up to about 30 mM, about 35 mM, about 40 mM, about 45 mM, about 50 mM, about 75 mM or about 100 mM. In one aspect, the formulation includes a buffer at a concentration of about 10 mM. In one aspect, the formulation includes a buffer at a concentration of about 15 mM. In one aspect, the formulation includes a buffer at a concentration of about 20 mM. In one aspect, the formulation includes a buffer at a concentration of about 30 mM. In one aspect, the formulation includes a buffer at a concentration of about 40 mM. In one aspect, the formulation includes a buffer at a concentration of about 50 mM. In one aspect, the buffer includes histidine/histidine hydrochloride. In one aspect, the buffer includes tris/tris hydrochloride. In one aspect, the buffer includes citrate. In one aspect, the buffer includes sodium acetate. In one aspect, the buffer includes phosphate. In one aspect, the buffer includes from about 10 mM to about 50 mM histidine/histidine HCl. In one aspect, the buffer includes from about 10 mM to about 30 mM histidine/histidine HCl. In one aspect, the buffer includes from about 10 mM to about 15 mM histidine/histidine HCl. In one aspect, the buffer includes from about 15 mM to about 20 mM histidine/histidine HCl. In one aspect, the buffer includes from about 20 mM to about 30 mM histidine/histidine HCl. In one aspect, the buffer includes about 5 mM histidine/histidine HCl. In one aspect, the buffer includes about 10 mM histidine/histidine HCl. In one aspect, the buffer includes about 15 mM histidine/histidine HCl. In one aspect, the buffer includes about 20 mM histidine/histidine HCl. In one aspect, the buffer includes about 25 mM histidine/histidine HCl. In one aspect, the buffer includes about 30 mM histidine/histidine HCl.

In one aspect, a stable pharmaceutical formulation is provided that includes a bioactive agent and D-α-Tocopheryl polyethylene glycol succinate (TPGS) as a surfactant. In one aspect, a stable pharmaceutical formulation is provided that includes from about 0.1 mg/mL to about 300 mg/mL bioactive agent, and from about 0.01% to about 0.1% TPGS. In one aspect, a stable pharmaceutical formulation is provided that includes from about 0.1 mg/mL to about 300 mg/mL bioactive agent, from about 0.01% to about 0.1% TPGS, and from about 10 mM to about 50 mM buffer. In one aspect, a stable pharmaceutical formulation is provided that includes from about 0.1 mg/mL to about 300 mg/mL bioactive agent, from about 0.01% to about 0.1% TPGS, and from about 10 mM to about 50 mM histidine/histidine HCl. In one aspect, a stable pharmaceutical formulation is provided that includes from about 0.1 mg/mL to about 300 mg/mL bioactive agent, from about 0.01% to about 0.1% TPGS, and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a stable pharmaceutical formulation is provided that includes from about 10 mg/mL to about 200 mg/mL of an antibody or antigen-binding antibody fragment, and from about to about 0.1% TPGS. In one aspect, a stable pharmaceutical formulation is provided that includes from about 10 mg/mL to about 200 mg/mL of an antibody or an antigen-binding antibody fragment, from about 0.01% to about 0.1% TPGS, and from about 10 mM to about 50 mM buffer. In one aspect, a stable pharmaceutical formulation is provided that includes from about 10 mg/mL to about 200 mg/mL of an antibody or an antigen-binding antibody fragment, from about 0.01% to about 0.1% TPGS, and from about 10 mM to about 50 mM histidine/histidine HCl. In one aspect, a stable pharmaceutical formulation is provided that includes from about 10 mg/mL to about 200 mg/mL of an antibody or an antigen-binding antibody fragment, about 0.02% TPGS, and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a stable pharmaceutical formulation is provided that includes from about 0.1 mg/mL to about 50 mg/mL of a therapeutic protein, and from about 0.01% to about 0.1% TPGS. In one aspect, a stable pharmaceutical formulation is provided that includes from about 0.1 mg/mL to about 50 mg/mL of a therapeutic protein, from about 0.01% to about 0.1% TPGS, and from about 10 mM to about 50 mM buffer. In one aspect, a stable pharmaceutical formulation is provided that includes from about 0.1 mg/mL to about 50 mg/mL of a therapeutic protein, from about 0.01% to about 0.1% TPGS, and from about 10 mM to about 50 mM histidine/histidine HCl. In one aspect, a stable pharmaceutical formulation is provided that includes from about 10 mg/mL to about 200 mg/mL of an antibody or an antigen-binding antibody fragment, about 0.02% TPGS, and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5. In one aspect, a stable pharmaceutical formulation is provided that includes a bioactive agent and D-α-Tocopheryl polyethylene glycol 1000 succinate (TPGS 1000) as a surfactant. In one aspect, a stable pharmaceutical formulation is provided that includes from about 0.1 mg/mL to about 300 mg/mL bioactive agent, and from about 0.01% to about 0.1% TPGS 1000. In one aspect, a stable pharmaceutical formulation is provided that includes from about 0.1 mg/mL to about 300 mg/mL bioactive agent, from about 0.01% to about 0.1% TPGS 1000, and from about mM to about 50 mM buffer. In one aspect, a stable pharmaceutical formulation is provided that includes from about 0.1 mg/mL to about 300 mg/mL bioactive agent, from about 0.01% to about TPGS 1000, and from about 10 mM to about 50 mM histidine/histidine HCl. In one aspect, a stable pharmaceutical formulation is provided that includes from about 0.1 mg/mL to about 300 mg/mL bioactive agent, from about 0.01% to about 0.1% TPGS 1000, and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a stable pharmaceutical formulation is provided that includes from about 10 mg/mL to about 200 mg/mL of an antibody or antigen-binding antibody fragment, and from about to about 0.1% TPGS 1000. In one aspect, a stable pharmaceutical formulation is provided that includes from about 10 mg/mL to about 200 mg/mL of an antibody or an antigen-binding antibody fragment, from about 0.01% to about 0.1% TPGS 1000, and from about 10 mM to about mM buffer. In one aspect, a stable pharmaceutical formulation is provided that includes from about 10 mg/mL to about 200 mg/mL of an antibody or an antigen-binding antibody fragment, from about 0.01% to about 0.1% TPGS 1000, and from about 10 mM to about 50 mM histidine/histidine HCl. In one aspect, a stable pharmaceutical formulation is provided that includes from about 10 mg/mL to about 200 mg/mL of an antibody or an antigen-binding antibody fragment, about 0.02% TPGS 1000, and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a stable pharmaceutical formulation is provided that includes from about 0.1 mg/mL to about 50 mg/mL of a therapeutic protein, and from about 0.01% to about 0.1% TPGS 1000. In one aspect, a stable pharmaceutical formulation is provided that includes from about 0.1 mg/mL to about 50 mg/mL of a therapeutic protein, from about 0.01% to about 0.1% TPGS 1000, and from about 10 mM to about 50 mM buffer. In one aspect, a stable pharmaceutical formulation is provided that includes from about 0.1 mg/mL to about 50 mg/mL of a therapeutic protein, from about 0.01% to about 0.1% TPGS 1000, and from about 10 mM to about 50 mM histidine/histidine HCl. In one aspect, a stable pharmaceutical formulation is provided that includes from about 10 mg/mL to about 200 mg/mL of an antibody or an antigen-binding antibody fragment, about 0.02% TPGS 1000, and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, the pharmaceutical formulation includes a tonicity agent. In one aspect, the tonicity agent includes a polyol, a saccharide, a carbohydrate, a salt, such as sodium chloride, or mixtures thereof. In one aspect, the polyol has a molecular weight that is less than about 600 kD, or from about 120 to about 400 kD. In one aspect, the tonicity agent is selected from mannitol, trehalose, sorbitol, erythritol, isomalt, lactitol, maltitol, xylitol, glycerol, lactitol, propylene glycol, polyethylene glycol, inositol, or mixtures thereof. In one aspect, the tonicity agent is a saccharide or carbohydrate selected from monosaccharides, disaccharides and polysaccharides or mixtures thereof. In one aspect, the saccharide or carbohydrate is selected from fructose, glucose, mannose, sucrose, sorbose, xylose, lactose, maltose, dextran, pullulan, dextrin, cyclodextrins, soluble starch, hydroxyethyl starch, water-soluble glucans, and mixtures thereof. In one aspect, the tonicity agent includes a reducing sugar or a non-reducing sugar or a mixture thereof.

In one aspect, the formulation includes the tonicity agent at a concentration from about 1 mg/ml to about 300 mg/ml, about 10 mg/ml to about 200 mg/ml, about 50 mg/ml to about 100 mg/ml, or about 80 mg/ml to about 90 mg/ml. In one aspect, the concentration of tonicity agent in the formulation is from about 1 mg/ml, about 5 mg/ml, about 10 mg/ml, about 15 mg/ml, about 20 mg/ml, about 25 mg/ml or about 50 mg/ml and up to about 100 mg/ml, about 150 mg/ml, about 200 mg/ml, about 250 mg/ml or about 300 mg/ml. In one aspect, the concentration of tonicity agent in the formulation is about 60 mg/ml, about 65 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, or about 100 mg/ml. In one aspect, the concentration of the tonicity agent is about 80 mg/ml. In one aspect, the concentration of the tonicity agent is about 81 mg/ml. In one aspect, the concentration of the tonicity agent is about 82 mg/ml. In one aspect, the concentration of the tonicity agent is about 83 mg/ml. In one aspect, the concentration of the tonicity agent is about 84 mg/ml. In one aspect, the concentration of the tonicity agent is about 85 mg/ml.

In one aspect, the formulation includes a non-reducing sugar as tonicity agent at a concentration from about 1 mg/ml to about 300 mg/ml, about 10 mg/ml to about 200 mg/ml, about 50 mg/ml to about 100 mg/ml, or about 80 mg/ml to about 90 mg/ml. In one aspect, the concentration of non-reducing sugar in the formulation is from about 1 mg/ml, about 5 mg/ml, about 10 mg/ml, about 15 mg/ml, about 20 mg/ml, about 25 mg/ml or about 50 mg/ml and up to about 100 mg/ml, about 150 mg/ml, about 200 mg/ml, about 250 mg/ml or about 300 mg/ml. In one aspect, the concentration of non-reducing sugar in the formulation is about 60 mg/ml, about 65 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, or about 100 mg/ml. In one aspect, the concentration of the non-reducing sugar is about 80 mg/ml. In one aspect, the concentration of the non-reducing sugar is about 81 mg/ml. In one aspect, the concentration of the non-reducing sugar is about 82 mg/ml. In one aspect, the concentration of the non-reducing sugar is about 83 mg/ml. In one aspect, the concentration of the non-reducing sugar is about 84 mg/ml. In one aspect, the concentration of the non-reducing sugar is about 85 mg/ml. In one aspect, the tonicity reagent includes a saccharide. In one aspect, the tonicity agent includes sucrose.

In one aspect, the formulation includes a salt as a tonicity agent. In one aspect, the salt is a pharmaceutically acceptable salt selected from sodium chloride, sodium succinate, sodium sulfate, potassium chloride, magnesium chloride, magnesium sulfate, calcium chloride, and mixtures thereof. In one aspect, the formulation includes a salt as a tonicity agent at a concentration from about 1 mg/ml to about 20 mg/ml. In one aspect, the salt is at a concentration from about 1 mg/ml, about 2 mg/ml, about 3 mg/ml, about 4 mg/ml, about 5 mg/ml, about 6 mg/ml, about 7 mg/ml, about 8 mg/ml, about 9 mg/ml or about 10 mg/ml and up to about 11 mg/ml, about 12 mg/ml, about 13 mg/ml, about 14 mg/ml, about 15 mg/ml, about 16 mg/ml, about 17 mg/ml, about 18 mg/ml, about 19 mg/ml or about 20 mg/ml. In one aspect, the salt is at a concentration of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml and about 20 mg/ml.

In one aspect, the pharmaceutical formulation includes an amino acid excipient. In one aspect, the pharmaceutical formulation includes an amino acid excipient to reduce protein aggregation. In one aspect, the pharmaceutical formulation includes a charged amino acid as a protein stabilizing excipient. In one aspect, the pharmaceutical formulation includes a positively charged amino acid as a stabilizing excipient, such as lysine, histidine or arginine. In one aspect, the pharmaceutical formulation includes a negatively charged amino acid, such as glutamic acid. In one aspect, the pharmaceutical formulation includes an uncharged polar amino acid such as serine, threonine or glutamine. In one aspect, the pharmaceutical formulation includes a hydrophobic amino acid such as alanine, valine, leucine, isoleucine, methionine or phenylalanine. In one aspect, a mixture of amino acids is included in the pharmaceutical formulation. In one aspect, the formulation includes a mixture of one or more of a positively charged amino acid, a negatively charged amino acid, an uncharged polar amino, and a hydrophobic amino acid as an excipient. In one aspect, the amino acid excipient is selected from arginine, cysteine, glycine, lysine, ornithine, proline or histidine. In one aspect, the amino acid excipient is selected from arginine, cysteine, glycine, or lysine. In one aspect, the amino acid is in the L-form. In one aspect, L-arginine hydrochloride (L-ArgHCl) is included in the pharmaceutical formulation. In one aspect, the pharmaceutical formulation includes from about 1 mg/ml to about 50 mg/ml, about 5 mg/ml to about 25 mg/ml, about 10 mg/ml to about 20 mg/ml, or about 1 mg/ml to about 10 mg/ml of at least one amino acid as an excipient. In one aspect, the pharmaceutical formulation includes from about 1 mg/ml, about 5 mg/ml, about 10 mg/ml, about 15 mg/ml, about 20 mg/ml or about 25 mg/ml and up to about 30 mg/ml, about 35 mg/ml, about 40 mg/ml, about 45 mg/ml or about 50 mg/ml of at least one amino acid as an excipient. In one aspect, the pharmaceutical formulation includes about 1 mg/ml, about 5 mg/ml, about 10 mg/ml, about 15 mg/ml, about 20 mg/ml, about mg/ml, about 30 mg/ml, about 35 mg/ml, about 40 mg/ml, about 45 mg/ml or about 50 mg/ml of at least one amino acid as an excipient.

In one aspect, the pharmaceutical formulation includes a chelating agent. In one aspect, the chelating agent is selected from aminopolycarboxylic acids, hydroxyaminocarboxylic acids, N-substituted glycines, 2-(2-amino-2-oxocthyl) aminoethane sulfonic acid (BES), deferoxamine (DEF), citric acid, niacinamide, and desoxycholates and mixtures thereof. In one aspect, the chelating agent is selected from ethylenediaminetetraacetic acid (EDTA), diethylenetriamine pentaacetic acid (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid (HIMDA), N,N-bis-hydroxyethylglycine (bicine) and N-(trishydroxymethylmethyl) glycine (tricine), glycylglycine, sodium desoxycholate, ethylenediamine; propylenediamine; diethylenetriamine; triethylenetetraamine (trien), ethylenediaminetetraaceto EDTA; disodium EDTA, calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In one aspect, the chelating agent is selected from salts of EDTA including dipotassium edetate, disodium edetate, edetate calcium disodium, sodium edetate, trisodium edetate, and potassium edetate; and a suitable salt of deferoxamine (DEF) is deferoxamine mesylate (DFM), or mixtures thereof. Chelating agents can be included in a pharmaceutical formulation as a free acid, free base form or salt form of the compound, or as an anhydrous, solvated or hydrated form of the compound. In one aspect, the pharmaceutical formulation includes a chelating agent at a concentration from about 0.01 mg/ml to about 50 mg/ml, from about 1 mg/ml to about 10 mg/ml, from about 1.5 mg/ml to about 5.0 mg/ml, or from about 0.1 mg/ml, about 0.5 mg/ml, about 1 mg/ml or about 5 mg/ml and up to about 10 mg/ml, about 20 mg/ml, about 30 mg/ml, about 40 mg/ml or about 50 mg/ml. In one aspect, the concentration of chelating agent is about 0.01 mg/ml, about 0.02 mg/ml, about 0.03 mg/ml, about mg/ml, about 0.05 mg/ml, about 0.06 mg/ml, about 0.07 mg/ml, about 0.08 mg/ml, about 0.09 mg/ml, or about 0.1 mg/ml. In one aspect, the concentration of chelating agent is about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, or about 1 mg/ml.

### Bioactive Agents

In one aspect, the pharmaceutical formulation includes a bioactive agent. In one aspect, the bioactive agent is a biotherapeutic. In one aspect, the bioactive agent includes a therapeutic macromolecule, for example, a polynucleotide or polypeptide. In one aspect, the bioactive agent includes a naturally occurring macromolecule. In one aspect, the bioactive agent includes a recombinantly produced macromolecule. In one aspect, the bioactive agent includes a polypeptide. In one aspect, the bioactive agent includes a polynucleotide. In one aspect, the bioactive agent includes a cell. In one aspect, the bioactive agent includes a polypeptide with a modified glycosylation profile. In one aspect, the bioactive agent includes an un-glycosylated polypeptide. In one aspect, the bioactive agent includes a chemically modified macromolecule, for example, a pegylated macromolecule or a macromolecule to which a diagnostic, targeting or therapeutic moiety has been appended. In one aspect, the bioactive agent is of therapeutic, scientific or commercial interest.

In one aspect, the bioactive agent includes an antibody or an antigen-binding fragment of an antibody. In one aspect, the bioactive agent includes an antibody-like protein. In one aspect, the bioactive agent includes a therapeutic antibody or antigen-binding antibody fragment. As used herein, the terms “antibody” and “immunoglobulin” can be used interchangeably and refer to a polypeptide or group of polypeptides that include at least one binding domain that is formed from the folding of polypeptide chains having three-dimensional binding spaces with internal surface shapes and charge distributions complementary to the features of an antigenic determinant of an antigen. A naturally occurring antibody typically has a tetrameric form, with two pairs of polypeptide chains, each pair having one “light” and one “heavy” chain. The variable regions of each light/heavy chain pair form an antibody binding site. Typically, each light chain is linked to a heavy chain by one covalent disulfide bond although the number of disulfide linkages between the heavy chains of different immunoglobulin isotypes can vary. Each heavy and light chain also has regularly spaced intrachain disulfide bridges. Each heavy chain has a variable domain (VH) at one end and constant domains (CH) at the other end. Each light chain has a variable domain at one end (VL) and a constant domain (CL) at the other end. The constant domain of the light chain is typically aligned with the first constant domain of the heavy chain, and the light chain variable domain is aligned with the variable domain of the heavy chain. Light chains can be classified as either lambda chains or kappa chains based on the amino acid sequence of the light chain constant region. The variable domain of a kappa light chain may also be denoted as V_{K}. Similarly, the variable domain of a lambda light chain may also be denoted as V_{λ}.

Bioactive agents can include immunoglobulin molecules of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1 m(f, z, a or x), G2m(n), G3m(g, b, or c), Am, Em, and Km(1, 2 or 3)). In one aspect, the bioactive agent is an antibody, an antigen-binding antibody fragment or an antibody-like molecule, such as a monoclonal antibody, for example, a full-length monoclonal antibody; a polyclonal antibody; a multispecific antibody that includes at least two different epitope binding fragments, for example, a bispecific antibody; a human antibody; a humanized antibody; a camelised antibody; a chimeric antibody; an anti-idiotypic (anti-Id) antibody; an intrabody; a diabody; a peptibody; a linear antibody; a single-chain antibody molecule; or an antigen binding fragment thereof. The terms “antigen-binding fragment” or “immunologically active fragment” refer to fragments of an antibody that contain at least one antigen-binding site and retain the ability to specifically bind to an antigen. Examples of antibody fragments that can be recombinantly produced include, but are not limited to, antibody fragments that include variable heavy- and light-chain domains, such as Fv, single-chain Fvs (scFv), single-chain antibodies, Fab fragments, Fab′ fragments, F(ab′)2 fragments. Antibody fragments can also include epitope-binding fragments or derivatives of any of the antibodies enumerated above. In one aspect, the antibody is part of a fusion protein. In one aspect, the antibody is a covalently modified antibody. The antibody may be murine, rat, human, or any other origin and can include chimeric and humanized antibodies. In one aspect, the antibody is an antibody fragment that retains one or more functional characteristics of the original antibody such as ligand binding or antagonist or agonist activity.

In one aspect, the bioactive agent includes the fragment crystallizable (Fc) region or domain of an IgG, which includes a paired set of antibody heavy chain domains, each of which include a heavy chain constant domain 2 (CH2) and a heavy chain constant domain 3 (CH3), which form a structure of about 50 kDa. The Fc region interacts with cell surface receptors called Fc receptors and some proteins of the complement system, allowing an antibody to activate the immune system.

In one aspect, the bioactive agent includes an antibody-drug conjugate (ADC) or an unconjugated antibody intermediate that is used to make an ADC. As used herein, “unconjugated antibody intermediate” refers to an antibody, for example, a monoclonal antibody, to which a biologically active molecule can be conjugated to form the ADC. Typically, the biologically active molecule is conjugated to the antibody intermediate through a linker. In one aspect, the linker is cleavable. In one aspect, the linker is non-cleavable. In one aspect, the biologically active molecule is conjugated to the antibody intermediate using site-specific conjugation. In one aspect, the biologically active molecule is conjugated to the antibody intermediate using non-site-specific conjugation. There are generally six types of biologically active molecules that are used in making ADC: small molecules, for example, highly potent cytotoxic agents that can be used to kill tumor cells; protein toxins, such as Pseudomonas or diphtheria toxin, that can be used to kill tumor cells; cytolytic immunomodulatory proteins, such as Fas ligand, that can be used to kill targeted cells; biologically active peptides, such as GLP-1, that can be used to extend the pharmacological half-life of the antibody; enzymes, such as urease, that can be used to modify the biochemistry of the targeted microenvironment; and radionuclides, such as 90 Y or ¹¹¹In, that can be used either to kill or image tumor cells.

In one aspect, the biologically active molecule conjugated to the antibody intermediate is a cytotoxic agent. As used herein, the term “cytotoxic agent” refers to a molecule that is detrimental to the growth, viability or propagation of cells. Examples of cytotoxic agents include, but are not limited to, mitomycin, doxorubicin, aminopterin, actinomycin, bleomycin, 9-amino-camptothecin, N⁸-acetyl spermidine, 1-(2-chloroethyl)-1,2-dimethanesulfonyl hydrazide, tallysomycin, cytarabine, etoposide, camptothecin, taxol, esperamicin, Podophyllotoxin, anguidine, vincristine, vinblastine, morpholine-doxorubicin, n-(5,5-diacetoxy-pentyl) doxorubicin, and derivatives thereof. Other cytotoxic agents are known and include, for example, those listed in Sapra et al., (2013) “Monoclonal antibody-based therapies in cancer: advances and challenges”. Pharmacol. & Therapeutics, 138:452-469.

In one aspect, the ADC includes a monoclonal antibody that specifically binds to a cell surface antigen. In one aspect, the ADC includes a monoclonal antibody that specifically binds to a tumor-associated antigen. In one aspect, the tumor-associated antigen is selected from HER2, EGFR, CD70 (CD27L), CD33, CD19, Mesothelin, CD22, CEACAMS, Trop-2 (M1S1, TACSTD2 or GA733-1), PSMA, CD37, CD30, DLL3, GPNMB, CD79b, GCC, NaPi2b, CA6, CD74, CD138, BCMA, myeloma antigen, SLAMF7 (CS1), CD56, ENPP3 (CD203c), TF (CD142), TIM1, FOLR1, MUC16 (CA-125), CanAg, Ckit (CD117 or SCFR), EphA2, Nectin 4(PVRL4), SLTRK6, HGFR (cMet), FGFR2, C4.41 (LYPD3), p-Cadherin (Cadherin 3), 5T4 (TPBG), S rEAP1, PTK7, Ephrin-A4 (EFNA4) LIV1 (SLC39A6 or ZIP6), TENB2, ETBR, integrin V3, crypto antigen, AGS-5 (SLC44A4), LY6E, AXL (UFO), CD205, CD25 (IL-2R alpha), LAMP-1 or MN/CAIX.

In one aspect, the ADC includes a biologically active agent conjugated to a monoclonal antibody through a linker. In one aspect, the linker is non-cleavable. In one aspect, the linker is cleavable. Generally, cleavable linkers exploit the differences in intracellular pH, reduction potential or enzyme concentration to trigger the release of the biologically active agent in the target cell. Cleavable linkers can include chemically labile linkers such as acid-cleavable linkers or reducible linkers; and enzyme-cleavable linkers such as peptide-based linkers or (3-glucuronide linkers. Cleavable linkers include, but are not limited to, linkers that include cleavable hydrazone, disulfide, peptide or thioether bonds. For example, cleavable linkers can include linker chemistries, including, but not limited to, hydrazone and hydrazide moieties, disulfide containing linkers such as N-succinimidyl-4-(2-pyridyldithio)pentanoate (SPP) and N-succinimidyl-4-(2-pyridyldithio)butyrate (SPDB), 4-(4′-acetylphenoxy)butanoic acid (AcBut) linkers, dipeptides valine-citrulline (Val-Cit) and phenylalanine-lysine (Phe-Lys) type linkers. Non-cleavable linkers rely on lysosomal proteolytic degradation of the antibody after internalization to release the biologically active molecule.

ADC present unique formulation challenges. Conjugation of a biologically active molecule to an antibody intermediate results in a molecule (the ADC) that can have different properties from the unconjugated antibody intermediate. For example, an antibody that exhibits acceptable aggregation behavior in its unconjugated form may behave differently in its conjugated form, for example, because of changes in surface properties or because the conjugate has altered the higher-order structure of the antibody. Although non-ionic surfactants such as polysorbate 80, polysorbate 20 and poloxamer 188 are widely used in biotherapeutic formulations to protect against aggregation and particle formation, these non-ionic surfactants are not easily removed from formulations, for example, using processes such as diafiltration. This can be problematic in the case of antibody intermediates used to generate antibody drug conjugates (ADC) because the presence of non-ionic surfactants may interfere with the conjugation process. Consequently, non-ionic surfactants such as polysorbate 80, polysorbate 20, and poloxamer 188 are often avoided in antibody intermediate formulations, despite the fact that formulations that lack a surfactant may be prone to particle formation. Advantageously, it has been found that inclusion of TPGS in an unconjugated antibody intermediate formulation does not interfere with the ADC conjugation process. In one aspect, a formulation is provided that includes an unconjugated antibody intermediate and TPGS as a surfactant.

In one aspect, the bioactive agent is an unconjugated antibody intermediate. In one aspect, the bioactive agent is a monoclonal antibody intermediate for use in forming an antibody drug conjugate (ADC). In one aspect, the bioactive agent is an antibody intermediate that specifically targets a tumour antigen that can be used to form an antibody drug conjugate (ADC) with a cytotoxic agent for use as an anti-cancer therapeutic.

In one aspect, the bioactive agent includes a therapeutic protein. In one aspect, the bioactive agent includes a naturally occurring therapeutic protein. In one aspect, the bioactive agent includes a recombinantly produced therapeutic protein. In one aspect, the bioactive agent includes a therapeutic protein such as an enzyme or enzymatically active polypeptide, a soluble receptor or receptor ligand, hormone, neurotransmitter, growth factor, integrin, interferon, an antigen, a secreted protein, or a fragment thereof. In one aspect, the therapeutic protein includes a fusion protein or a proteolysis targeting chimera (protac). In one aspect, the bioactive agent includes a therapeutic peptide. As used herein “therapeutic peptide” refers to a bioactive polypeptide that includes less than about 500, about 250, about 100, about 50 or about 20 amino acids and has a molecular weight below about 100 kDa, or about 50 kDa.

In one aspect, the bioactive agent includes cells. In one aspect, the bioactive agent includes cells for use in cellular therapy. In one aspect, the bioactive agent includes whole, viable cells such as stem cells, progenitor cells or differentiated cells. In one aspect, the cells include pluripotent cells. In one aspect, the cells include multipotent cells. Examples of cells suitable for use in cellular therapy include, but are not limited to, hematopoietic stem cells (HSC), skeletal muscle stem cells, mesenchymal stem cells, lymphocytes, T-cells, dendritic cells, and pancreatic islet cells. In one aspect, the cells originate from the subject to be treated (i.e., autologous cells). In one aspect, the cells originate from a donor (i.e., allogeneic cells).

In one aspect, the bioactive agent includes a therapeutic polynucleotide, for example, a single-stranded or double-stranded polynucleotide such as deoxyribonucleotides (DNA), for example, cDNA or ribonucleotides (RNA) including, for example, RNA-based therapeutics such as antisense RNA, microRNAs (miRNAs), shorthairpin RNAs (shRNAs), RNA interference (RNAi), small interfering RNA (siRNA) and ribozymes. In one aspect, the bioactive agent includes plasmid DNA or an expression vector. In one aspect, the bioactive agent includes a plasmid, phagemid, cosmid or yeast artificial chromosomes (YAC). In one aspect, the bioactive agent includes a viral vector, including for example, retroviral, adenoviral, poxviral (e.g., vaccinia), adeno-associated viral (AAV), Newcastle disease viral (NDV) or a herpes simplex viral vector.

In one aspect, a pharmaceutical formulation is provided that includes an antibody or antigen binding fragment thereof. In one aspect, a pharmaceutical formulation is provided that includes a monoclonal antibody or antigen binding fragment thereof that specifically binds to IL-5Rα. The term “specifically binds” refers to the ability of an antibody or antigen binding fragment to recognize and bind to a target antigen, set of antigens or a domain or amino acid sequence within a target antigen with no or insignificant binding to other molecules in the sample. In one aspect, a pharmaceutical formulation is provided that includes Benralizumab or an antigen binding fragment thereof. As used herein, the term “Benralizumab”, also known by its trade name Fasenra™ (MedImmune), refers to a human IgG antibody that binds IL-5Rα.

In one aspect, a pharmaceutical formulation is provided that includes a monoclonal antibody that specifically binds to IL-5Rα. In one aspect, a stable pharmaceutical formulation is provided that includes from about 10 mg/mL to about 200 mg/mL of a monoclonal antibody or antigen-binding antibody fragment that specifically binds to IL-5Rα; and from about 0.01% to about 0.1% TPGS. In one aspect, a stable pharmaceutical formulation is provided that includes from about 10 mg/mL to about 200 mg/mL Benralizumab; and from about 0.01% to about 0.1% TPGS.

In one aspect, a stable pharmaceutical formulation is provided that includes from about 10 mg/mL to about 200 mg/mL of a monoclonal antibody or antigen-binding antibody fragment that specifically binds to IL-5Rα; from about 0.01% to about 0.1% TPGS; and from about 10 mM to about 50 mM buffer. In one aspect, a stable pharmaceutical formulation is provided that includes from about 10 mg/mL to about 200 mg/mL of a monoclonal antibody or antigen-binding antibody fragment that specifically binds to IL-5Rα; from about 0.01% to about 0.1% TPGS; and from about 10 mM to about 50 mM histidine/histidine HCl. In one aspect, a stable pharmaceutical formulation is provided that includes from about 10 mg/mL to about 200 mg/mL of a monoclonal antibody or antigen-binding antibody fragment that specifically binds to IL-5Rα; about 0.02% TPGS; and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a stable pharmaceutical formulation is provided that includes from about 10 mg/mL to about 200 mg/mL Benralizumab; from about 0.01% to about 0.1% TPGS; and from about 10 mM to about 50 mM buffer. In one aspect, a stable pharmaceutical formulation is provided that includes from about 10 mg/mL to about 200 mg/mL Benralizumab; from about 0.01% to about 0.1% TPGS; and from about 10 mM to about 50 mM histidine/histidine HCl. In one aspect, a stable pharmaceutical formulation is provided that includes from about 10 mg/mL to about 200 mg/mL Benralizumab; about 0.02% TPGS; and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a pharmaceutical formulation is provided that includes a monoclonal antibody that specifically binds to IL-5Rα. In one aspect, a stable pharmaceutical formulation is provided that includes from about 10 mg/mL to about 200 mg/mL of a monoclonal antibody or antigen-binding antibody fragment that specifically binds to IL-5Rα; and from about 0.01% to about 0.1% TPGS 1000. In one aspect, a stable pharmaceutical formulation is provided that includes from about 10 mg/mL to about 200 mg/mL Benralizumab; and from about 0.01% to about TPGS 1000.

In one aspect, a stable pharmaceutical formulation is provided that includes from about 10 mg/mL to about 200 mg/mL of a monoclonal antibody or antigen-binding antibody fragment that specifically binds to IL-5Rα; from about 0.01% to about 0.1% TPGS 1000; and from about 10 mM to about 50 mM buffer. In one aspect, a stable pharmaceutical formulation is provided that includes from about 10 mg/mL to about 200 mg/mL of a monoclonal antibody or antigen-binding antibody fragment that specifically binds to IL-5Rα; from about 0.01% to about 0.1% TPGS 1000; and from about 10 mM to about 50 mM histidine/histidine HCl. In one aspect, a stable pharmaceutical formulation is provided that includes from about 10 mg/mL to about 200 mg/mL of a monoclonal antibody or antigen-binding antibody fragment that specifically binds to IL-5Rα; about 0.02% TPGS 1000; and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a stable pharmaceutical formulation is provided that includes from about 10 mg/mL to about 200 mg/mL Benralizumab; from about 0.01% to about 0.1% TPGS 1000; and from about 10 mM to about 50 mM buffer. In one aspect, a stable pharmaceutical formulation is provided that includes from about 10 mg/mL to about 200 mg/mL Benralizumab; from about 0.01% to about 0.1% TPGS 1000; and from about 10 mM to about 50 mM histidine/histidine HCl. In one aspect, a stable pharmaceutical formulation is provided that includes from about 10 mg/mL to about 200 mg/mL Benralizumab; about 0.02% TPGS 1000; and about 20 mM histidine/histidine HCl. In one aspect, the pharmaceutical formulation has a pH from about 3 to about 9. In one aspect, the pharmaceutical formulation has a pH from about 4 to about 8. In one aspect, the pharmaceutical formulation has a pH from about 5.5 to about 7.5.

In one aspect, a method for preventing, treating, or managing an eosinophil mediated disorder or disease or one or more symptoms thereof, is provided. In one aspect, the method includes administering to a subject a pharmaceutical formulation that includes an antibody that specifically binds to human interleukin-5 receptor (IL-5Rα) and D-α-Tocopheryl polyethylene glycol 1000 succinate (TPGS 1000) as surfactant. In one aspect, the subject suffers from chronic obstructive pulmonary disease (COPD). In one aspect, the subject suffers from mild persistent or mild intermittent asthma.

### Method of Use

In one aspect, a method of treating or preventing a disease or disorder is provided. In one aspect, the method includes administering to a subject in need thereof a therapeutically effective amount of a pharmaceutical formulation that includes a bioactive agent and a surfactant that includes D-α-Tocopheryl polyethylene glycol succinate (TPGS) as described herein. In one aspect, the method includes administering to a subject in need thereof a therapeutically effective amount of a pharmaceutical formulation that includes a bioactive agent and a surfactant that includes D-α-Tocopheryl polyethylene glycol 1000 succinate (TPGS 1000). In one aspect, a pharmaceutical formulation that includes a bioactive agent and TPGS is provided for use as a medicament. In one aspect, a pharmaceutical formulation that includes a bioactive agent and TPGS is provided for the preparation of a medicament for treating a disease or disorder in a subject. In one aspect, the pharmaceutical formulation is a liquid, for example, an aqueous solution. In one aspect, the pharmaceutical formulation is a lyophilized powder that can be reconstituted into a suitable solution for administration by adding an appropriate diluent.

In one aspect, the subject is selected from a rodent (such as a mouse or rat), rabbit, a companion animal (such as a cat, dog and horse), a farm animal (such as a cow, sheep, pig and goat), or a primate (such as a human or non-human primate). In one aspect, the subject is a human.

In one aspect, the pharmaceutical formulation is administered parenterally. In one aspect, the pharmaceutical formulation is administered intravenously, intramuscularly or subcutaneously. In one aspect, the pharmaceutical formulation is administered intravenously as a bolus or as continuous infusion over a period of time. Suitable devices for parenteral administration include needles (including microneedles, microprojections, soluble needles and other micropore formation techniques), injectors, needle-free injectors and other infusion techniques.

### Article of Manufacture

In one aspect, an article of manufacture is provided. In one aspect, the article of manufacture includes a device or container that contains a pharmaceutical formulation that includes a bioactive agent and a surfactant that includes D-α-Tocopheryl polyethylene glycol succinate (TPGS) as described herein. In one aspect, the article of manufacture includes a device or container that contains a pharmaceutical formulation that includes a bioactive agent and a surfactant that includes D-α-Tocopheryl polyethylene glycol 1000 succinate (TPGS 1000). In one aspect, the container or device is a syringe, for example, a pre-filled syringe; an auto injector; bottle; vial; or test tube. In one aspect, the article of manufacture includes a device or container that contains the pharmaceutical formulation and a label on, or associated with, the device or container that provides directions for use. In one aspect, the article of manufacture further includes other materials desirable from a commercial or user standpoint, including buffers, diluents, filters, needles, syringes, or package inserts with instructions for use.

In one aspect, a kit is provided. In one aspect, the kit includes at least one device or container that contains a pharmaceutical formulation that includes a bioactive agent and a surfactant that includes D-α-Tocopheryl polyethylene glycol succinate (TPGS) as described herein. In one aspect, the kit includes at least one device or container that contains a pharmaceutical formulation as described herein and an injection device. In one aspect, the injection device is adapted for intravenous, intramuscular or subcutaneous administration. In one aspect, the kit includes instructions for administration of the composition.

### Incorporation by Reference

All references cited herein, including patents, patent applications, papers, text books and the like, and the references cited therein, to the extent that they are not already, are hereby incorporated herein by reference in their entirety.

### EXAMPLES

### Example 1— Surfactant Screening

In this example, the stability of four different antibodies was evaluated using three different surfactants and a no-surfactant control.
(mAb-1) a human anti-Ang2-TNFa bispecific antibody;
(mAb-2) a bivalent, bispecific human IgG1κ monoclonal antibody that binds to both the Pseudomonas aeruginosa PcrV type III secretion (T3S) protein involved in host cell cytotoxicity and the Psl exopolysaccharide involved in Psuedomonas aeruginosa colonization and tissue adherence;
(mAb-3) an anti-interleukin 13 IgG1 monoclonal antibody with YTE amino acid substitutions in the crystallizable fragment (Fc) domain; and
(mAb-4) an anti-influenza A IgG1 monoclonal antibody that specifically binds to influenza A hemagglutinin stalk.

Each were formulated in 3 cc glass vials (1.1 mL volume) as described in the table below.

| | | | | Molecule |
|---|---|---|---|---|
| Molecule | Formulation | Concentration | | type |
| mAb-1 | 25 mM His/His-HCl, | 50 | mg/mL | Bi-specific |
| | 235 mM Sucrose, pH 6 | | | mAb |
| mAb-2 | 25 mM His/His-HCl, 8% | 100 | mg/mL | Bi-specific |
| | Glycine, 4% Arg-HCl, pH 6 | | | mAb |
| mAb-3 | 25 mM His/His-HCl, | 150 | mg/mL | mAb IgG1 |
| | 190 mM Arg-HCl, pH 6.0 | | | |
| mAb-4 | 25 mM His/His-HCl | 50 | mg/ml | mAb IgG1 |
| | 235 mM Sucrose, pH 6.0 | | | |

In separate 1.1 mL aliquots of the formulations containing test molecules, 0.02% TPGS (Vitamin E succinate with PEG-1000), 0.02% PS80 (polysorbate 80), 0.02% Poloxamer 188 (P188), 0.02% Kolliphor HS15 (Polyoxyl 15 Hydroxystearate), 0.02% Kolliphor EL (PEG-35 Castor Oil), 0.02% Kolliphor RH40 (PEG-40 Hydrogenated Castor Oil), or 1% PEG 300 (Polyethylene glycol 300) were added. A separate vial in which no surfactant was added was used as a control. Vials were subjected to three freeze-thaw cycles (3FT), and a shipping simulation procedure or vortex shaking for 2.5 h, respectively. Test molecule stability was assessed by visual inspection, micro-flow imaging (MFI), and high-pressure size exclusion chromatography (HPSEC).

Visual inspection of the samples was performed by examining the samples in their respective container for particles, color, and clarity using appropriate standards.

Detection of non-visible particles was performed using Micro Flow Imaging (MFI) using the 5200 series Micro-Flow Imaging system (available from Protein Simple) in accordance with the supplier's instructions.

HPSEC was performed using an Agilent HPLC system (Santa Clara, CA) with a TSK-Gel G3000 column for analysis. Briefly, 100 μg of antibody was loaded on the column by injecting 25 μl of the antibody diluted to 10 mg/ml in phosphate buffered saline. Standard integration parameters were used for automatic integration of aggregate, monomer and fragment peaks.

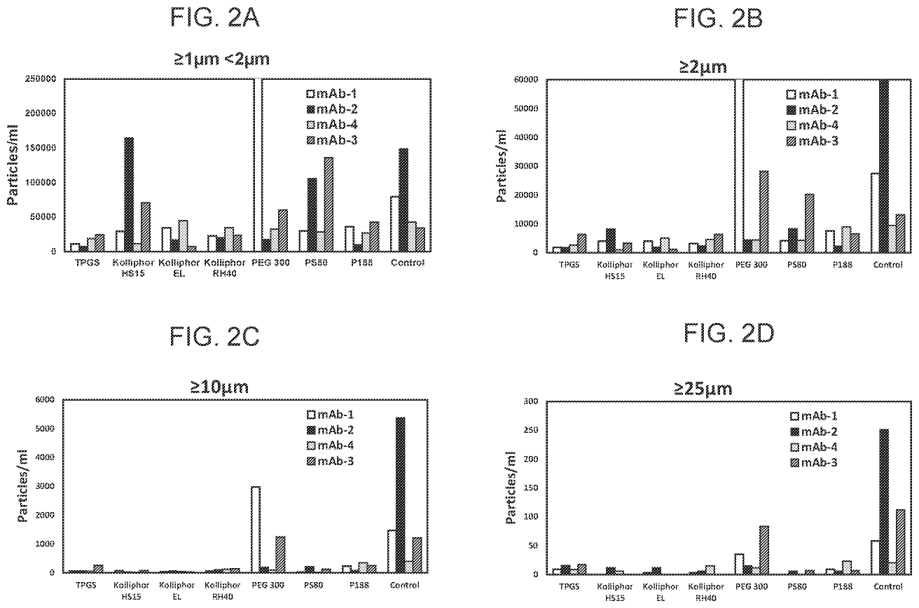

Test molecule stability was assessed by visual inspection, MFI, and HPSEC. Results from time zero (T0) and post shipping simulation (SS) are shown in FIGS. 2 (MFI) and 3 (HPSEC) as examples.

In the initial stability screen for the test molecules formulated in TPGS, Kolliphor HS15, Kolliphor EL, Kolliphor RH40, PEG 300, PS80, P188, or no surfactant (control), the MFI results in FIG. 2 demonstrate that among all particle sizes assessed (≥1 μm≤2 μm, ≥2 μm, ≥10 μm, and ≥25 μm), the control group containing no surfactant consistently displayed the highest numbers of particles/mL compared to TGPS, PS80, and P188. The performance of the individual surfactants varied depending on the size of particle assessed. TPGS showed consistently low amounts of particles/mL compared to other surfactants across the entire range of particle sizes, most notably in the ≥1 μm≤2 μm and ≥2 μm categories.

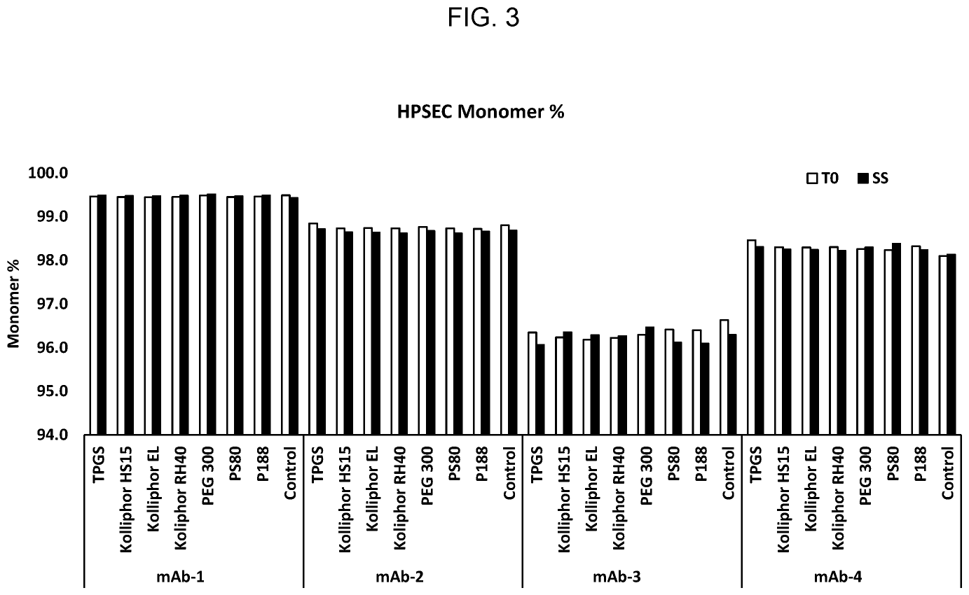

FIG. 3 shows the percentage of monomers (a metric of stability, or non-aggregation) of each test molecule at TO or after SS as measured by HPSEC. Based on these data, for all test molecules, the formulations containing different surfactant types showed comparable HPSEC monomer percentages (% monomer) compared to the control.

### Example 2— Stressed, Accelerated and Long-Term Stability

To determine the effects of different surfactants on the stressed, accelerated and long term stability of the four test molecules, formulations of mAb-1, mAb-2, mAb-3, and mAb-4 at a concentration range of 50-150 mg/mL were spiked with 0.02% TPGS, PS80, or P188 and stored in 3 cc glass vials (with 1.3 mL fill) and stored at four different temperatures: 40° C., 25° C., 2-8° C., and −80° C. The stability of the formulations of test molecules containing surfactants was assessed via visual inspection, MFI, and HPSEC.

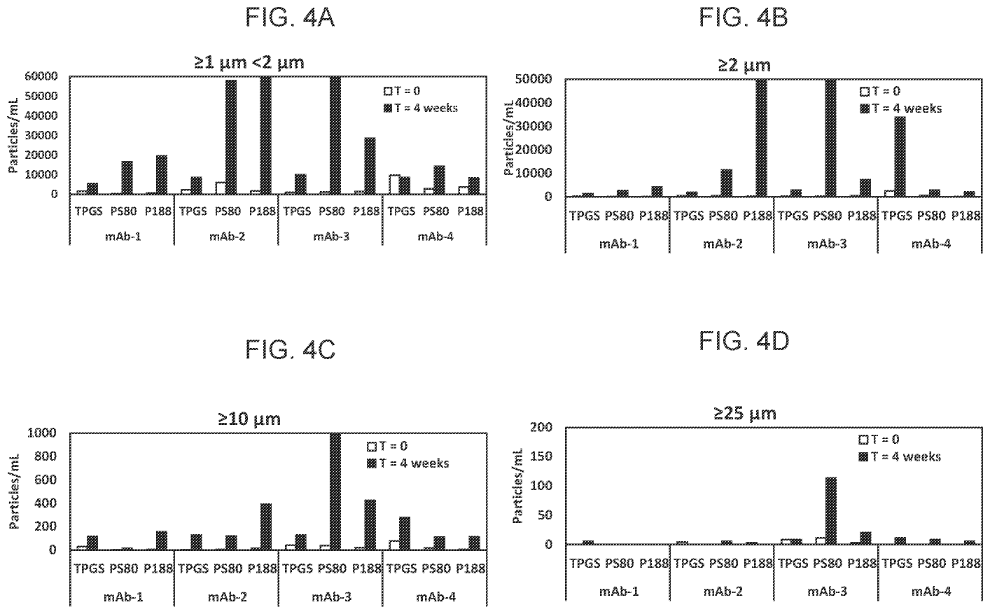

Test molecule stability was assessed by visual inspection, MFI, and HPSEC. Results are reported in FIGS. 4-11.

In FIG. 4A-D, the stressed stability of mAb-1, mAb-2, mAb-3, and mAb-4 formulated with TPGS, PS80, and P188 is shown as assessed by MFI after storage at 40° C. for 4 weeks. Most notably, mAb-3 appears to be less stable (high particles/mL at all measured sizes) when formulated with PS80 under these conditions, while all four of the test molecules are stable when formulated with TPGS.

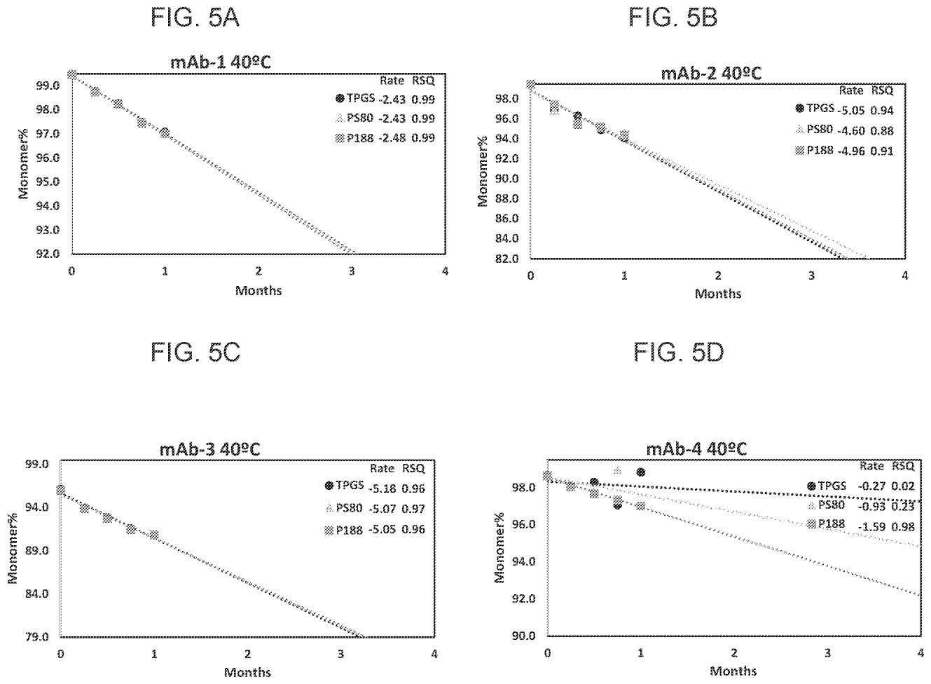

FIG. 5A-D show HPSEC data for mAb-1, mAb-2, mAb-3, and mAb-4 formulated with TPGS, PS80, and P188 and stored at 40° C. over a period of 0-4 weeks. mAb-4 formulated in TPGS shows minimal % monomer loss (0.27% loss per month) after 4 weeks stored at 40° C. (reported as % monomers) compared to approximately 1% and 1.6% monomer loss per month when formulated in PS80 and P188, respectively. The type of surfactant used in the formulation of mAb-1, mAb-2, and mAb-3 did not appear to significantly affect the decrease in % monomers present in the solution after 4 weeks storage.

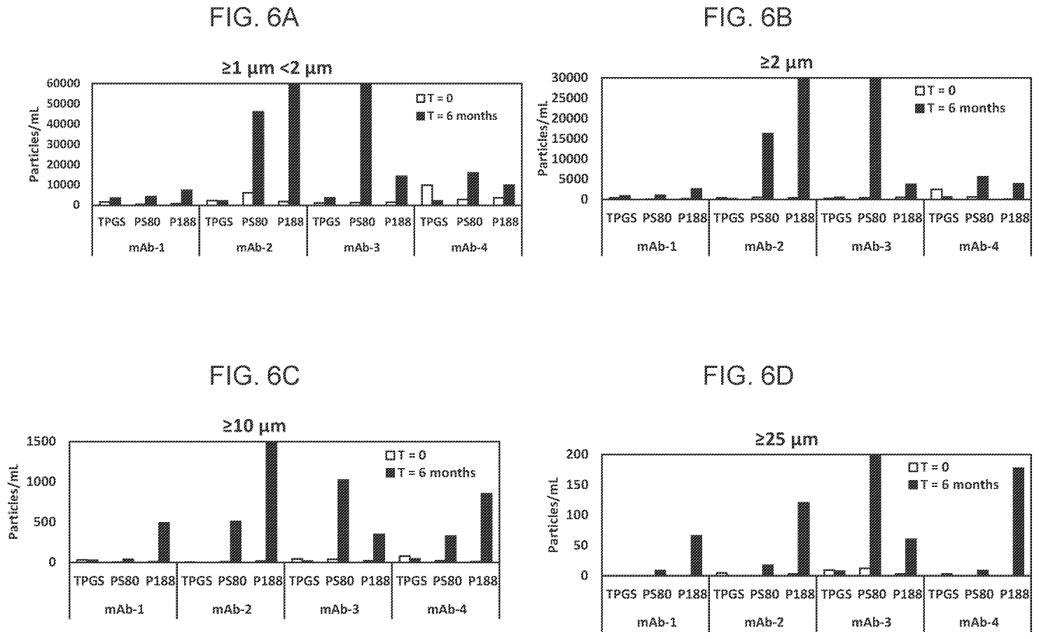

In FIG. 6A-D, the accelerated stability of mAb-1, mAb-2, mAb-3, and mAb-4 formulated with TPGS, PS80, and P188 is shown as assessed by MFI after storage at 25° C. for 6 months. As in FIG. 4, mAb-3 appears to be highly unstable when formulated with PS80 under these conditions, while all four of the test molecules are highly stable when formulated with TPGS. Decreased levels of stability were also observed with P188, most notably mAb-2 and mAb-4.

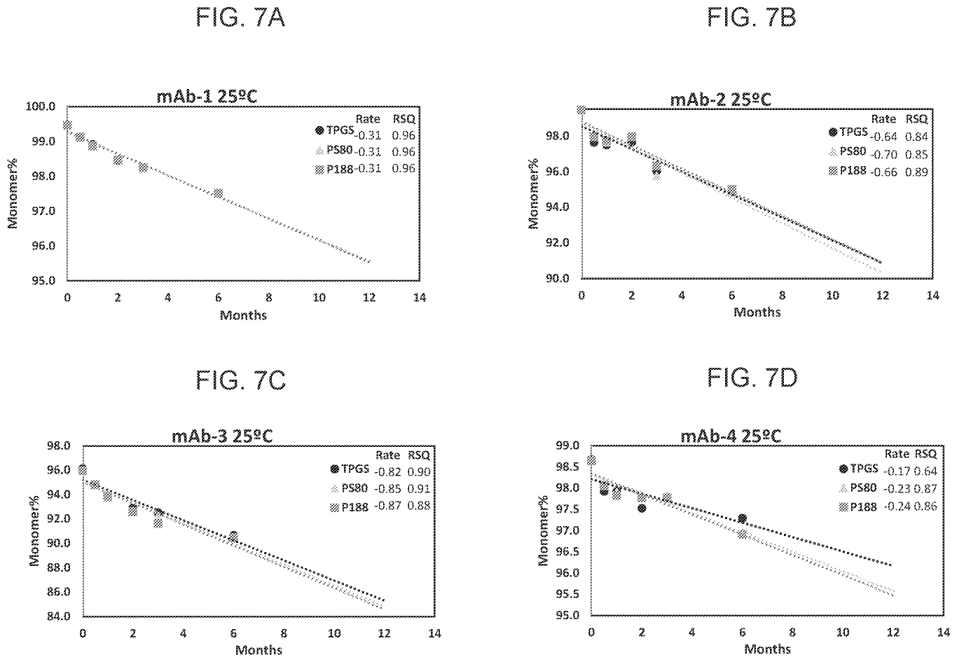

In FIG. 7A-D, HPSEC data are shown for mAb-1, mAb-2, mAb-3 and mAb-4 formulated with TPGS, PS80, and P188 and stored at 25° C. over a period of 0-6 months. The type of surfactant used in the formulation of mAb-1, mAb-2, mAb-3, and mAb-4 did not appear to significantly affect % monomers present in the solution at 6 months.

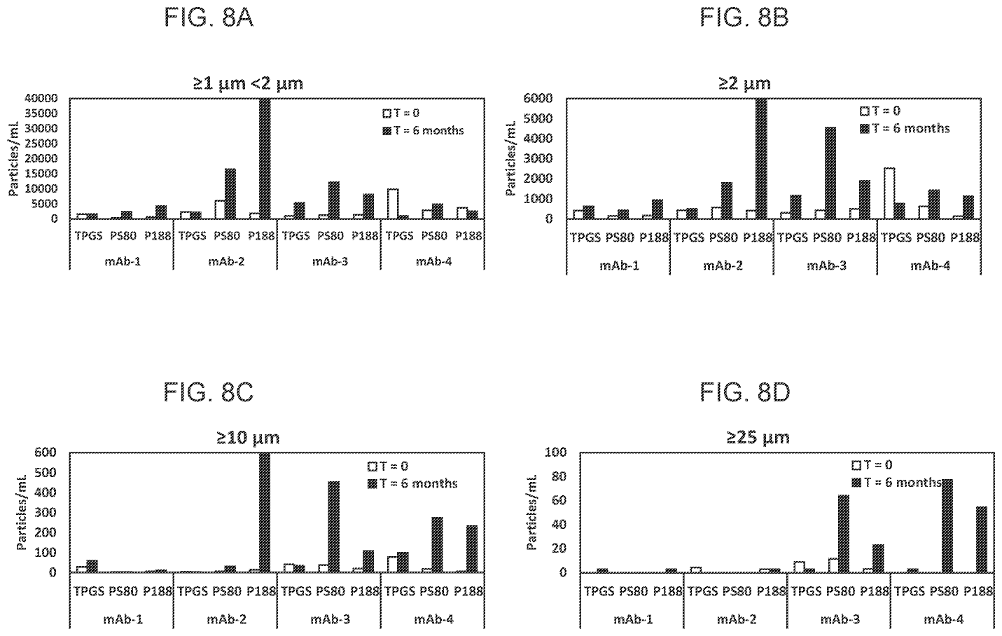

In FIG. 8A-D, MFI data are shown for mAb-1, mAb-2, mAb-3, and mAb-4 formulated with TPGS, PS80, and P188 after storage at 5° C. for 6 months. As in the previous Figures, the TPGS formulations of the four test molecules appeared to be more stable compared to the PS80 and P188 surfactant groups, especially for mAb-2 and mAb-3.

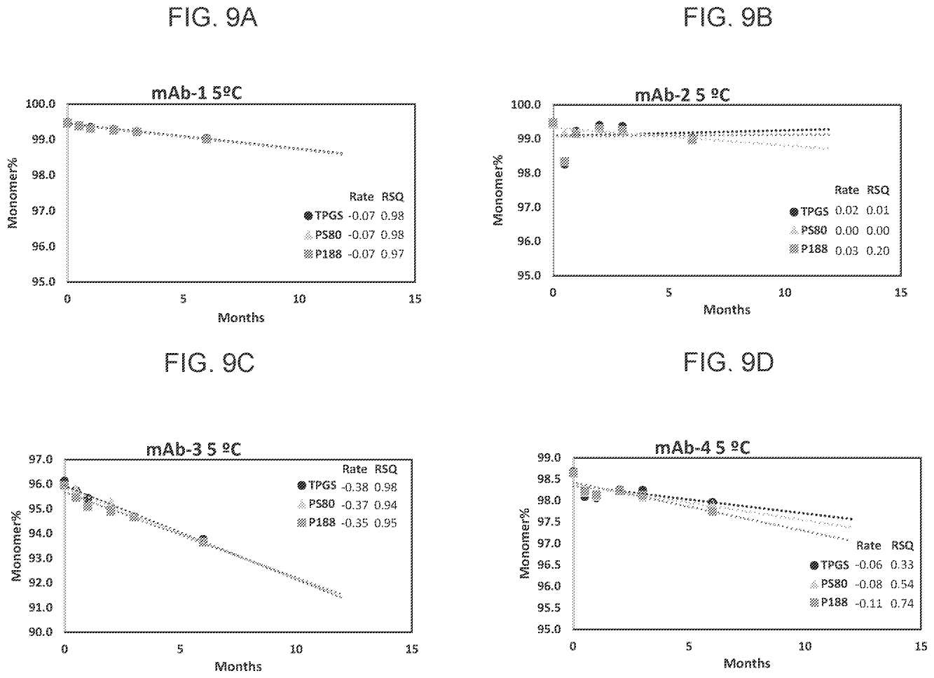

FIG. 9A-D shows the HPSEC data for mAb-1, mAb-2, mAb-3, and mAb-4 formulated with TPGS, PS80, and P188 after storage at 5° C. for 0-6 months. The TPGS formulations showed comparable stability to PS80 and P188.

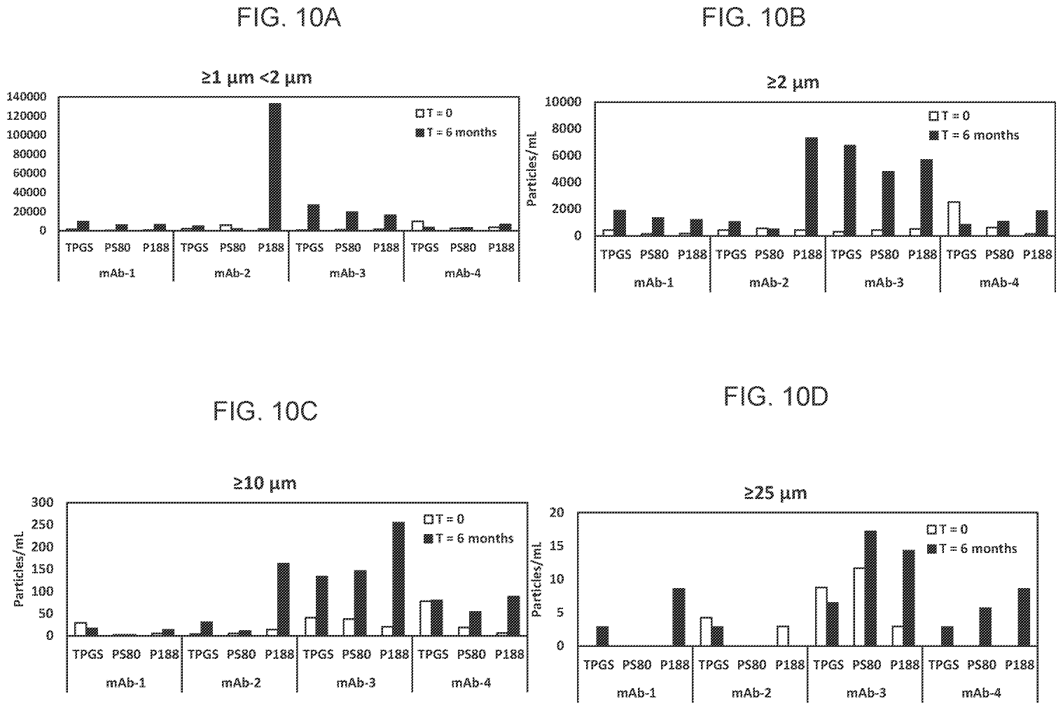

In FIG. 10A-D, the long-term stability of mAb-1, mAb-2, mAb-3, and mAb-4 formulated with TPGS, PS80, and P188 after storage at −80° C. for 6 months as assessed by MFI are shown. In each surfactant group, mAb-3 displayed a slightly higher number of particles/mL compared to other test molecules.

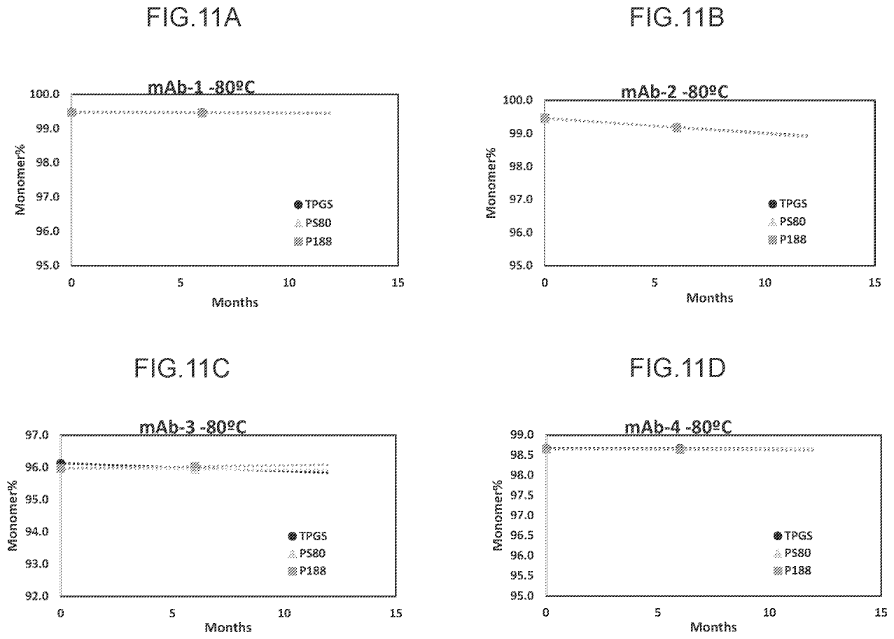

FIG. 11A-D show the HPSEC data for mAb-1, mAb-2, mAb-3, and mAb-4 formulated with TPGS, PS80, and P188 after storage at −80° C. for 0-6 months. The TPGS formulations showed comparable stability to PS80 and P188.

### Example 3— Concentration Range of TPGS Formulation for mAb-2 and mAb-4

To determine the effects of different concentrations of TPGS on stability, mAb-2 (100 mg/mL, formulated in 25 mM His-His/HCl, 8% Gly, 4% Arg-HCl) and mAb-4 (50 mg/mL, formulated in 25 mM His-His/HCl, 235, mM sucrose) were spiked with 0.001%, 0.01%, 0.02%, 0.10%, 1.00% or 5.00% of TPGS, 0.02% PS80, 0.02% PS20, or 0.02% P188 and stored in 3 cc glass vials with 1.3 mL fill. The formulations were subjected to three freeze-thaw cycles and shipping simulation (SS). Stability of the formulations was assessed via visual inspection, MFI, HPSEC, and Capillary Gel Electrophoresis (CGE).

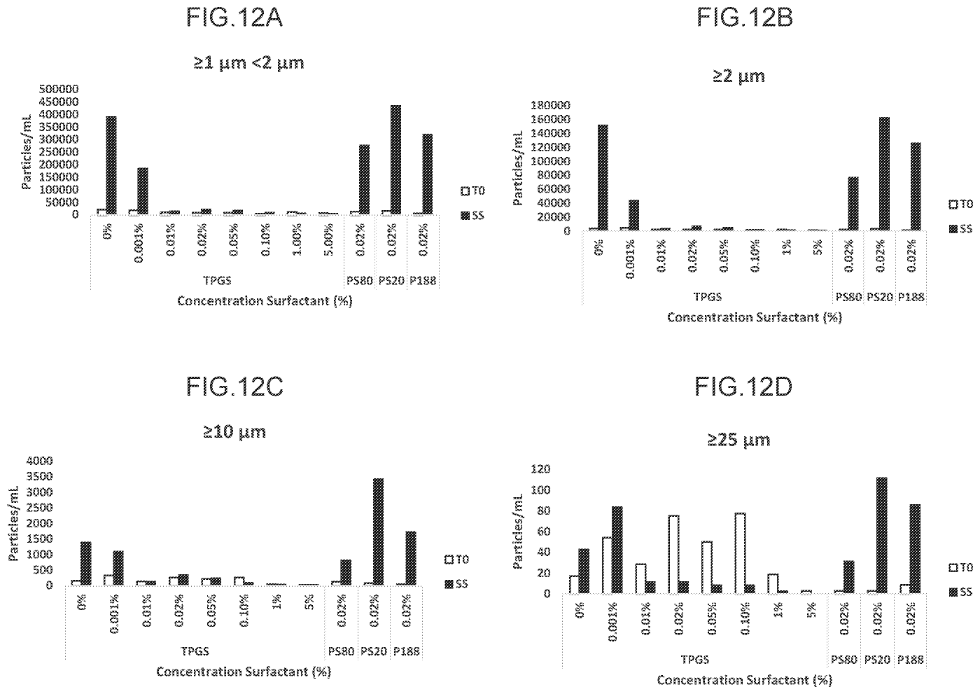

Test molecule stability was assessed by visual inspection, MFI, Horizon, HPSEC, and CGE. Results are reported in FIGS. 12-16.

In FIG. 12A-D, data comparing the stability of surfactant formulations of mAb-2 at time zero (T0) to post-shipping simulation (SS) as assessed via MFI are shown. TPGS started to show significant effect in reducing particles from a level as low as 0.01%. The particle count of the formulation with 0.01% TPGS is significantly less than formulations containing PS80, PS20 and P188 with a higher surfactant level (0.02%). The effect of TPGS was demonstrated at higher concentrations from 0.02% to 5.00%. All showed a reduced level of particles, which were significantly better than the other surfactants.

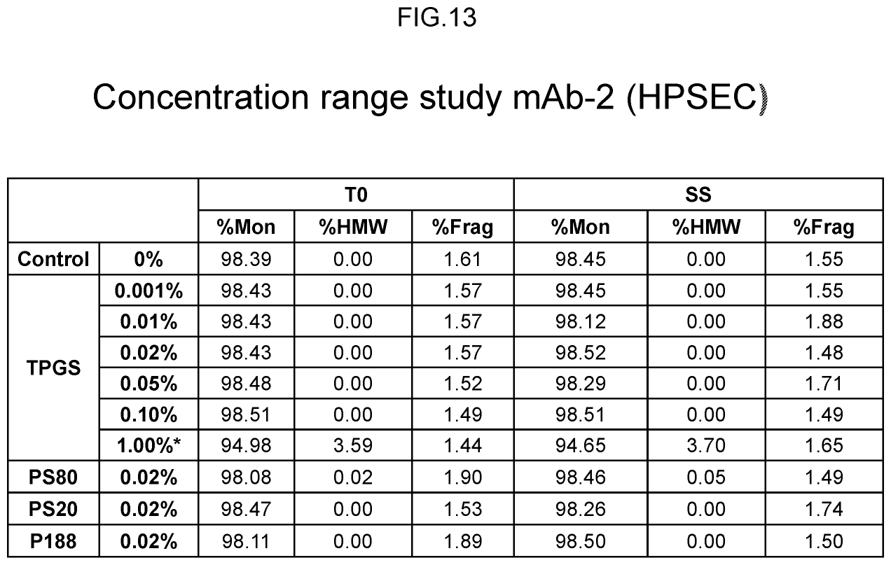

FIG. 13 shows data comparing the stability of surfactant formulations of mAb-2 at time zero (T0) to post-shipping simulation (SS) as assessed via HPSEC. The TPGS formulation ranges of 0.01-0.10% displayed comparable purity profiles among themselves and also to the formulations containing 0.02% of PS80, PS20 and P188. However, at the higher concentration of TPGS (1%), the percentage of monomers appeared to decrease with an increased level of % HMW. However, this apparent increase in % HMW is not, in fact, due to an increase in aggregation, but rather to interference of TPGS eluting at similar retention time.

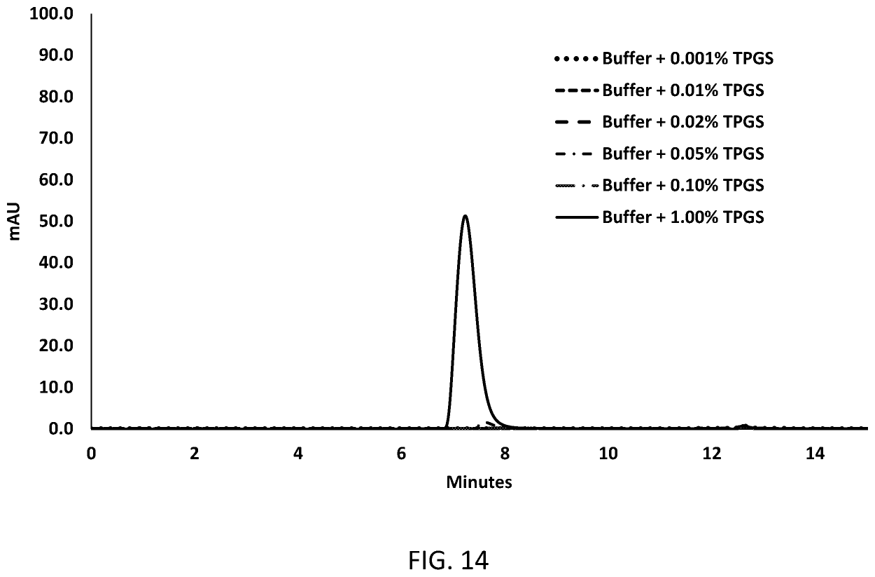

FIG. 14 shows overlaid HPSEC chromatograms of blank formulation buffer containing different levels of TPGS (from 0.001% to 1.00%). As the TPGS concentration increases, the area of the peak increases which results in an increased interference with the mAb chromatograms (e.g. 1.00% TPGS). At lower concentrations (e.g., 0.001% to 0.10%), the interreference from TPGS is negligible.

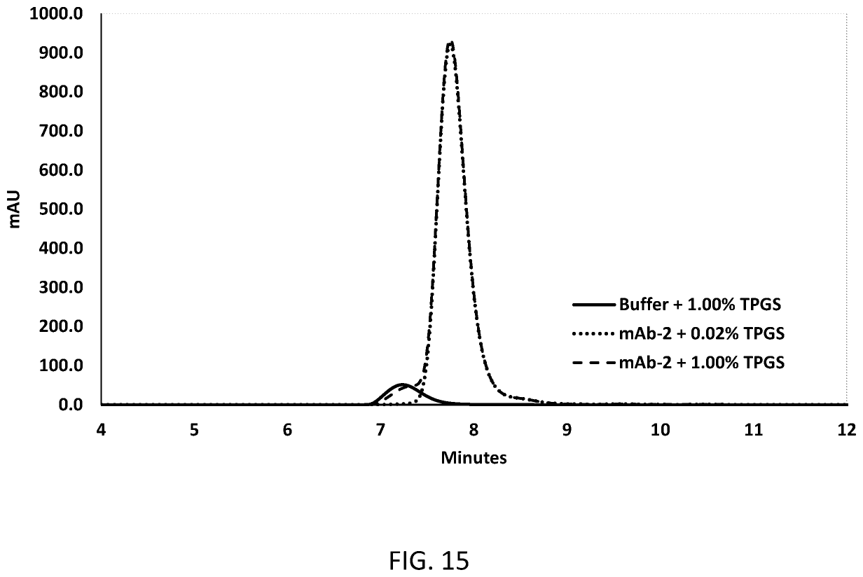

FIG. 15 shows overlaid HPSEC chromatograms of blank formulation buffer containing 1.00% TPGS with mAb-2 samples containing 0.02% or 1.00% of TPGS. The TPGS eluted at similar retention times as the aggregate peak of the mAb. When TPGS was used at 1.00%, it interfered with the mAb chromatograms leading to an overestimation of % HMW and an apparent decrease in % monomer.

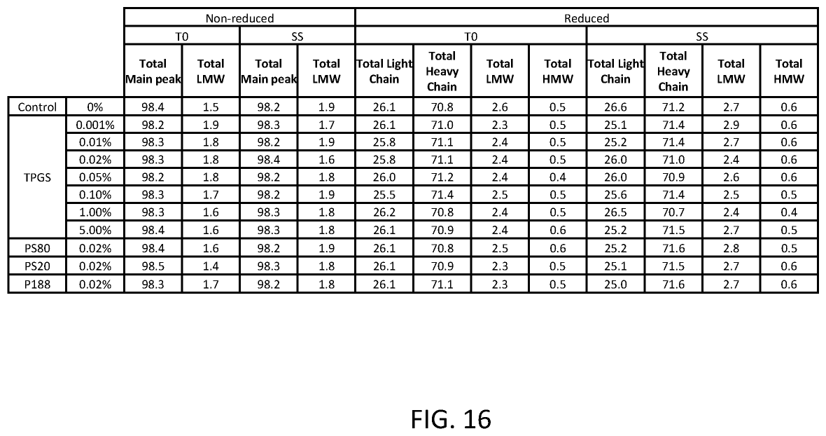

Capillary gel electrophoresis (CGE) was used to assess the stability of the surfactant formulations as an orthogonal method. As shown in FIG. 16, formulations containing different levels of TPGS (from 0.001% to 1.00%) all had similar values, and were similar to formulations containing 0.02% of PS80, PS20 and P188 in both the non-reduced and reduced conditions. The CGE data confirmed that the apparent reduction in % monomer for the 1.00% TPGS formulation observed by HPSEC was, in fact, due to interreference of TPGS and not aggregation.

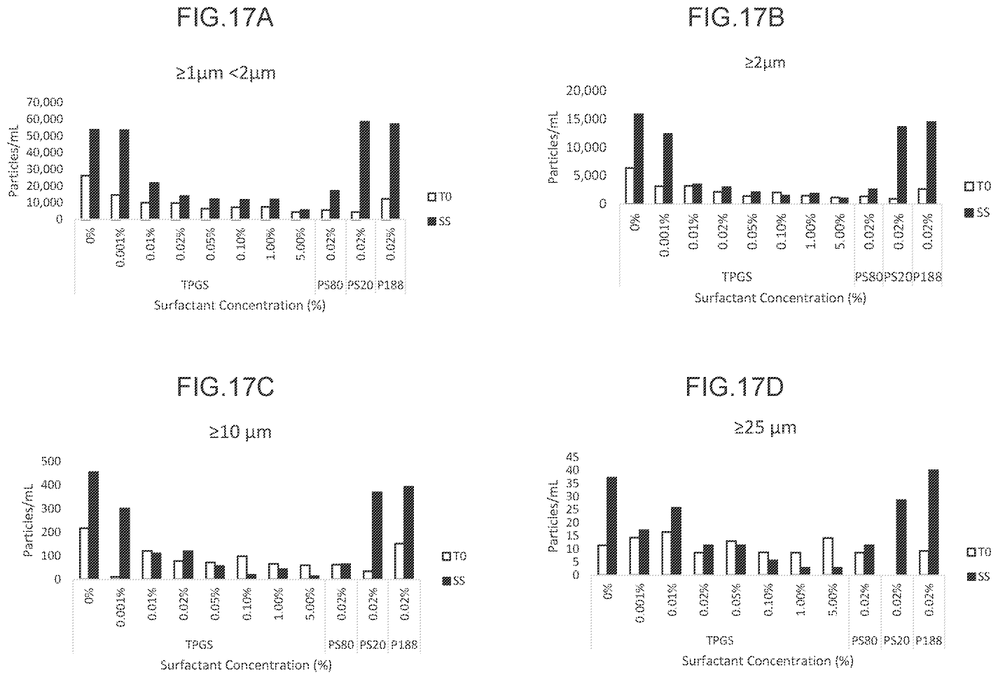

In FIG. 17A-D, data comparing the stability of surfactant formulations of mAb-4 at time zero (T0) to post-shipping simulation (SS) as assessed via MFI are shown. The presence of TPGS in the formulation reduced particles starting from 0.01% TPGS. At 0.02% TPGS, the formulations showed less or comparable particle counts compared to formulation containing PS80 at the same level, and significantly less particle counts than formulations containing PS20 or P188. As % TPGS was increased, the particle numbers decreased further showing a promising effect of TPGS overall in reducing particles.

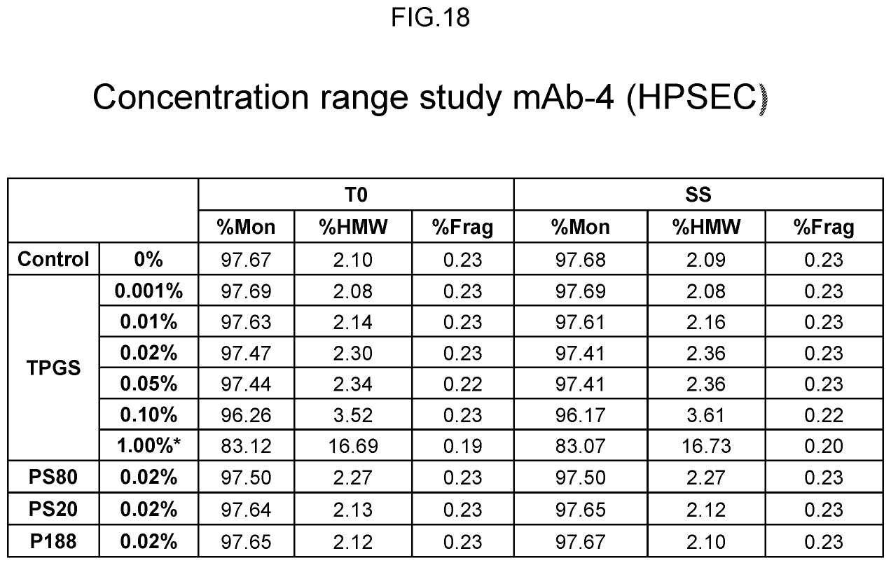

FIG. 18 shows data comparing the stability of surfactant formulations of mAb-4 at time zero (T0) to post-shipping simulation (SS) as assessed via HPSEC. Similar to mAb-2, the TPGS formulation ranges of 0.01-0.10% displayed comparable purity profiles among themselves and also to the formulations containing 0.02% of PS80, PS20 and P188. At 1.00% TPGS, the formulation appeared to show a decrease in % monomer. However, this was due to interreference of TPGS with the chromatogram, rather than an increase in aggregation.

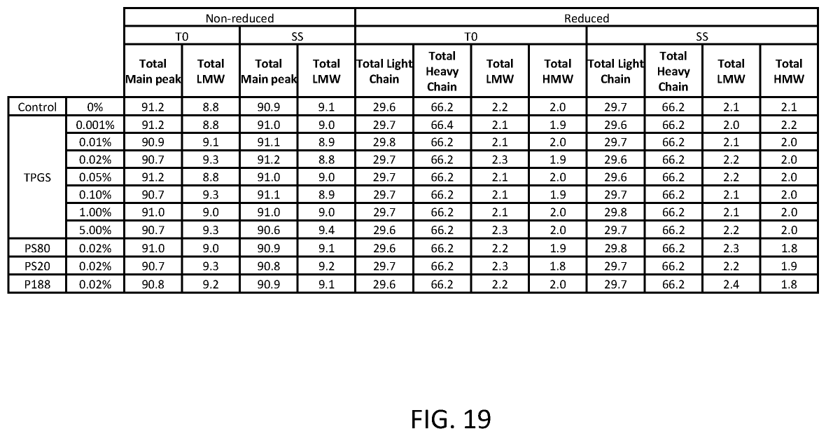

FIG. 19 shows the results from capillary gel electrophoresis (CGE) that was used to assess the stability of the surfactant formulations of mAb-4 and similar to mAb-2 above, formulations containing different levels of TPGS (from 0.001% to 1.00%) all showed similar values, and were similar to formulations containing 0.02% of PS80, PS20 and P188 in both non-reduced and reduced conditions.

### Example 4— Evaluation of Stability of mAb-5 and mAb-6 Formulated in TPGS

In this example, the stability of two antibodies was determined:
(mAb-5) a biparatopic monospecific antibody intermediate targeting HER2; and
(mAb-6) an antibody intermediate targeting B7H4.

To determine the stability of mAb-5 and mAb-6 (20 mg/mL, formulated in 20 mM His-His HCL, 240 mM sucrose, pH 6), C-Pak bags (30 mL) containing each test molecule were spiked with 0.02% TPGS. The formulations were subjected to three freeze-thaw (3xFT) cycles and shipping simulation. Stability of the formulations was assessed at time zero (T0), post-3xFT, and post-shipping simulation via visual inspection, MFI, HPSEC, and UV absorption at 280 nm (A280).

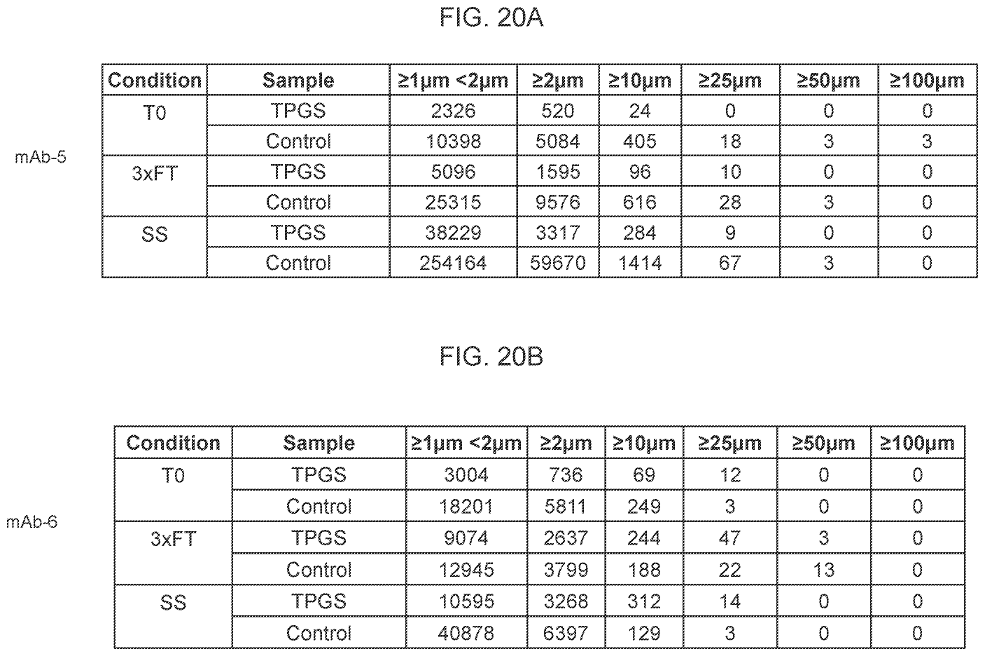

Stability of mAb-5 and mAb-6 were assessed by visual inspection, MFI, and HPSEC. Key results are reported in FIGS. 20-23.

FIG. 20A-B shows the comparative stability of test molecules mAb-5 and mAb-6 formulated in 0.02% TPGS or control at time zero (T0), after 3 freeze-thaw cycles (3xFT) and post-shipping simulation (SS) as measured by MFI. In each of the three time points for both test molecules, the TPGS sample showed a marked decrease in the number of particles compared to the control sample.

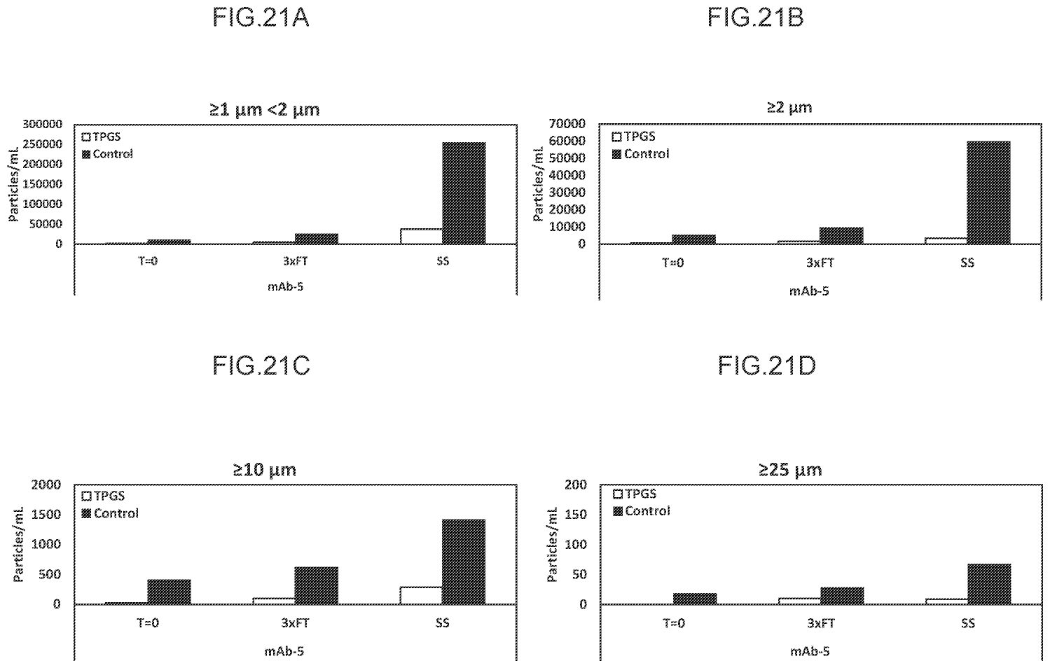

FIG. 21 represents the mAb-5 stability MFI data from FIG. 20A in graphical format.

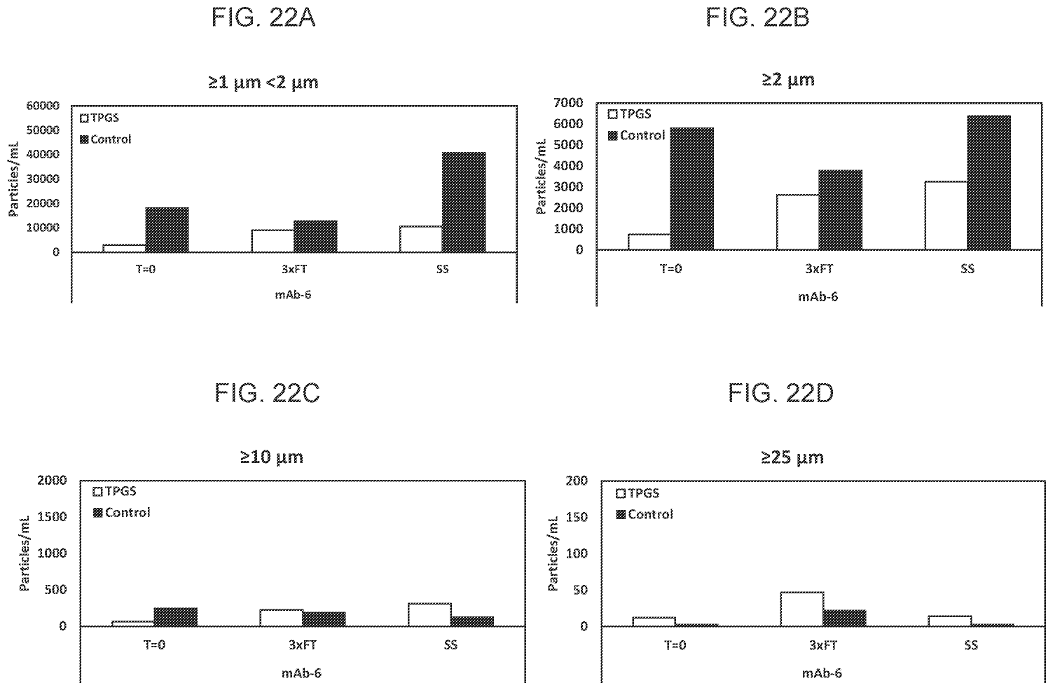

FIG. 22 represents the mAb-6 stability MFI data from FIG. 20B in graphical format.

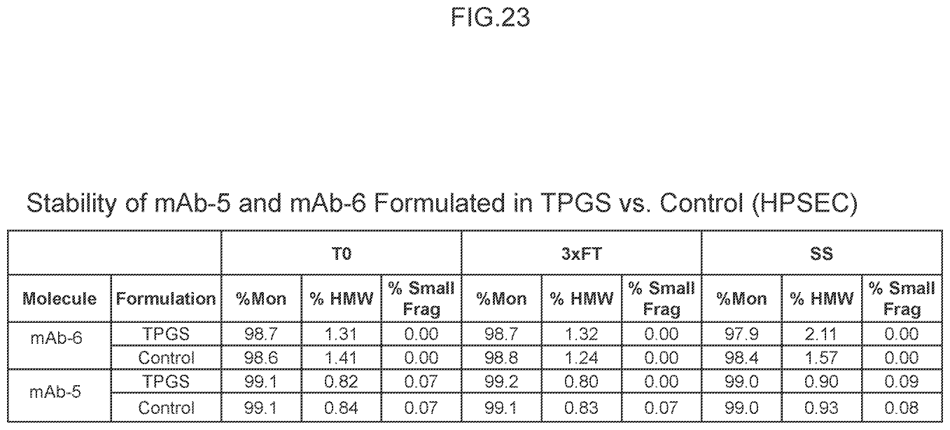

FIG. 23 shows the stability of mAb-5 and mAb-6 as measured by HPSEC at T0, after 3xFT, and post-SS. The presence of TPGS in the formulation of either test molecule did not appear to significantly influence the stability compared to control at any point in the study.

What is claimed is:

1. A stable pharmaceutical formulation comprising:
(a) a bioactive agent;
(b) a buffer; and
(c) a surfactant comprising D-α-Tocopheryl polyethylene glycol succinate (TPGS),
wherein the formulation has a pH from about 3 to about 9, about 4 to about 8 or about 5.5 to about 7.5.

2. The formulation of claim 1, comprising a liquid formulation or a lyophilized formulation.

3. The formulation of claim 1, wherein the bioactive agent comprises a therapeutic polypeptide.

4. The formulation of claim 3, wherein the therapeutic polypeptide is selected from an antibody or antigen-binding antibody fragment, an enzyme or enzymatically-active polypeptide, a soluble receptor or receptor ligand, hormone, neurotransmitter, growth factor, integrin, interferon or an antigen.

5. The formulation of claim 4, wherein the antibody or antigen-binding antibody fragment comprises a monoclonal antibody.

6. The formulation of claim 3 or 4, wherein the antibody or antigen-binding antibody fragment is selected from human, humanized, chimeric, multispecific, bispecific, Fab′, F(ab′)2, Fv, single chain Fv (scFv), diabodies, peptibodies, linear antibodies and single-chain antibodies.

7. The formulation of claim 3 or 4, wherein the therapeutic polypeptide comprises a fusion polypeptide, a proteolysis targeting chimera (protac), or an antibody-drug conjugate (ADC).

8. The formulation of claim 1, wherein the bioactive agent comprises a therapeutic polynucleotide.

9. The formulation of claim 8, wherein the therapeutic polynucleotide comprises single-stranded or double-stranded DNA or RNA.

10. The formulation of claim 8 or 9, wherein the therapeutic polynucleotide is selected from cDNA, antisense RNA, microRNA (miRNA), shorthairpin RNA (snRNA), RNA interference (RNAi), small interfering RNA (siRNA) and ribozymes.

11. The formulation of any of claims 8 to 10, wherein the therapeutic polynucleotide comprises a plasmid, phagemid, cosmid or yeast artificial chromosomes (YAC).

12. The formulation of any of claims 8 to 11, wherein the therapeutic polynucleotide comprises a retroviral, adenoviral, poxviral, adeno-associated viral (AAV), Newcastle disease viral (NDV) or herpes simplex viral vector.

13. The formulation of claim 1, wherein the bioactive agent comprises cells selected from stem cells, progenitor cells and differentiated cells.

14. The formulation of claim 1, wherein the formulation comprises the bioactive agent at a concentration from about 0.1 mg/mL to about 300 mg/mL.

15. The formulation of claim 14, wherein the bioactive agent comprises an antibody or antigen-binding antibody fragment with a concentration from about 10 mg/mL to about 200 mg/mL.

16. The formulation of claim 15, wherein the concentration of antibody or antigen-binding antibody fragment is at least about 10 mg/mL, about 15 mg/mL, about 20 mg/mL, about 25 mg/mL, about 50 mg/mL, about 75 mg/mL or about 100 mg/mL and up to about 125 mg/mL, about 150 mg/mL, about 175 mg/mL, about 200 mg/mL, 2 about 50 mg/mL or 3 about 00 mg/mL.

17. The formulation of claim 14, wherein the bioactive agent comprises a therapeutic protein with a concentration from about 0.1 mg/mL, about 0.5 mg/mL, about 1 mg/mL, about 2 mg/mL, about 3 mg/mL, about 4 mg/mL or about 5 mg/mL and up to about 10 mg/mL, about 15 mg/mL, about 20 mg/mL, about 25 mg/mL, about 30 mg/mL, about 35 mg/mL, about 35 mg/mL, about 40 mg/mL, about 45 mg/mL or about 50 mg/mL.

18. The formulation of any of the preceding claims, wherein the concentration of TPGS is from about 0.001% to about 1%.

19. The formulation of claim 18, wherein the concentration of TPGS is from about 0.001%, about 0.005%, about 0.01%, about 0.05% and up to about 0.1%, about 0.5% or about 1%.

20. The formulation of claim 18 or 19, wherein the concentration of TPGS is about 0.01%, about 0.02%, about 0.03%, about 0.04%, about 0.05%, about 0.06%, about 0.07%, about 0.08%, about 0.09% or about 0.1%.

21. The formulation of any of the preceding claims, wherein the buffer is selected from acetate, acetic acid, succinate, succinic acid, phosphate, phosphoric acid, ascorbate, ascorbic acid, lactate, lactic acid, tartartic acid, maleic acid, glycine, gluconate, citrate, histidine, imidazole, bicarbonate and carbonic acid, sodium benzoate, benzoic acid, edetate, malate, tris, glycylglycine and mixtures thereof.

22. The formulation of claim 21, wherein the buffer comprises histidine/histidine hydrochloride, tris/tris hydrochloride, citrate, sodium acetate, phosphate, or a combination thereof.

23. The formulation of the preceding claims, wherein the buffer has a concentration from about 0.1 mM to about 100 mM.

24. The formulation according to claim 23, wherein the buffer concentration is from about 0.1 mM, about 0.5 mM, about 1 mM, about 5 mM, about 10 mM, about 20 mM or about 25 mM and up to about 30 mM, about 35 mM, about 40 mM, about 45 mM, or about 50 mM.

25. The formulation according to claim 23 or 24, wherein the buffer comprises about 0.5, about 1 mM, about 5 mM, about 10 mM, about 20 mM, about 25 mM, about 30 mM, about 35 mM, about 40 mM, about 45 mM or about 50 mM histidine/histidine HCl.

26. The formulation of any of the preceding claims, further comprising a tonicity agent.

27. The formulation of claim 26, wherein the tonicity agent comprises a polyol, a saccharide, a carbohydrate, a salt, or a mixture thereof.

28. The formulation of claim 26 or 27, wherein the formulation includes the tonicity reagent at a concentration from about 1 mg/ml to about 300 mg/ml, about 10 mg/ml to about 200 mg/ml or about 50 mg/ml to about 100 mg/ml.

29. The formulation of any of claims 26 to 28, wherein the tonicity reagent comprises a saccharide at a concentration of about 80 mg/ml to about 90 mg/ml.

30. The formulation of any of claims 26 to 28, wherein the tonicity reagent comprises a salt at a concentration from about 1 mg/mL to about 20 mg/ml.

31. The formulation of any of the preceding claims, further comprising an amino acid as an excipient.

32. The formulation of claim 31, wherein the amino acid is selected from arginine, cysteine, glycine, lysine, glycine, ornithine, proline, alanine, glutamine, glutamic acid, histidine, or a salt or combination thereof.

33. The formulation of any of the preceding claims, further comprising a chelating agent.

34. The formulation of claim 17, wherein the chelating agent is selected from aminopolycarboxylic acids, hydroxyaminocarboxylic acids, N-substituted glycines, 2-(2-amino-2-oxocthyl) aminoethane sulfonic acid (BES), deferoxamine (DEF), citric acid, niacinamide, and desoxycholates and mixtures thereof.

35. The formulation of claim 17, wherein the chelating agent is selected from ethylenediaminetetraacetic acid (EDTA), diethylenetriamine pentaacetic acid (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid N,N-bis-hydroxyethylglycine (bicine) and N-(trishydroxymethylmethyl) glycine (tricine), glycylglycine, sodium desoxycholate, ethylenediamine; propylenediamine; diethylenetriamine; triethylenetetraamine (trien), ethylenediaminetetraaceto EDTA; disodium EDTA, calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof.

36. The formulation of claim 33 or 34, wherein the concentration of the chelating agent is from about 0.01 mg/ml to about 50 mg/ml.

37. A stable pharmaceutical formulation comprising:
(a) from about 0.1 mg/mL to about 300 mg/mL bioactive agent;
(b) from about 10 mM to about 50 mM buffer;
(c) from about 0.01% to about 0.1% TPGS,
wherein the formulation has a pH from about 3 to about 9.

38. The formulation of claim 37, wherein the buffer comprises histidine/histidine HCl.

39. The formulation of claim 38, comprising from about 20 mM to about 30 mM histidine/histidine HCl.

40. The formulation of claim 37, wherein the formulation has a pH from about 4 to about 8.

41. The formulation of claim 37, wherein the formulation has a pH from about 5.5 to about 7.5.

42. The formulation of claim 37, comprising from about 10 mg/mL to about 200 mg/mL of an antibody or antigen-binding antibody fragment.

43. The formulation of claim 37, comprising from about 0.1 mg/mL to about 50 mg/mL of a therapeutic protein.

44. The formulation of any of the preceding claims, comprising less than about 10,000, about about 1,000, about 750, about 500, about 250, about 150, about 100, or about 50 particles greater than about 2 μm, about 5 μm, about 10 μm, about 15 μm, about 20 μm or about 25 μm diameter/mL.

45. The formulation of any of the preceding claims, wherein the formulation is stable at a temperature of about 40° C. for up to about 3 months.

46. The formulation of any of the preceding claims, wherein the formulation is stable at a temperature of about 40° C. for up to about 6 months.

47. The formulation of any of the preceding claims, wherein the formulation is stable at a temperature of about 25° C. for up to about 6 months.

48. The formulation of any of the preceding claims, wherein the formulation is stable at a temperature of about 25° C. for up to about 12 months.

49. The formulation of any of the preceding claims, wherein the formulation is stable at a temperature of about 2° C. to about 8° C. for up to about 12 months.

50. The formulation of any of the preceding claims, wherein the formulation is stable at a temperature of about 2° C. to about 8° C. for up to about 24 months.

51. The formulation of any of the preceding claims, wherein the formulation is stable at a temperature of about 2° C. to about 8° C. for up to about 36 months.

52. The formulation of any of the preceding claims, wherein the formulation is stable at a temperature of about −20° C. for up to about 6 months.

53. The formulation of any of the preceding claims, wherein the formulation is stable at a temperature of about −20° C. for up to about 12 months.

54. The formulation of any of the preceding claims, wherein the formulation is stable at a temperature of about −20° C. for up to 2 about 4 months.

55. The formulation of any of the preceding claims, wherein the formulation is stable at a temperature of about −20° C. for up to about 36 months.

56. The formulation of any of the preceding claims, wherein the formulation is stable at a temperature of about −80° C. for up to about 6 months.

57. The formulation of any of the preceding claims, wherein the formulation is stable at a temperature of about −80° C. for up to about 12 months.

58. The formulation of any of the preceding claims, wherein the formulation is stable at a temperature of about −80° C. for up to about 24 months.

59. The formulation of any of the preceding claims, wherein the formulation is stable at a temperature of about −80° C. for up to about 36 months.

60. A method of reducing particle formation in an aqueous pharmaceutical formulation, the method comprising adding D-α-Tocopheryl polyethylene glycol 1000 succinate (TPGS) to the formulation, wherein less than about 10,000, about 5,000, about 1,000, about 750, about 500, about 250, about 150, about 100, or about 50 particles greater than about 2 μm, about 5 μm, about 10 μm, about 15 μm, about 20 μm or about 25 μm diameter/mL are formed.

61. The method according to claim 60, wherein the pharmaceutical formulation comprises less than about 5,000 particles greater than about 2 μm in diameter/mL.

62. The method according to claim 60, wherein the pharmaceutical formulation comprises less than about 1,000 particles greater than about 2 μm in diameter/mL.

63. The method according to claim 60, wherein the pharmaceutical formulation comprises less than about 6,000 particles greater than about 10 μm in diameter/mL.

64. The method according to claim 60, wherein the pharmaceutical formulation comprises less than about 600 particles greater than about 25 μm in diameter/mL.

65. The method according to claim 60, wherein D-α-Tocopheryl polyethylene glycol 1000 succinate (TPGS) is added to the formulation in an amount ranging from about 0.001% to about 1%.

66. A method of reducing interference during antibody-drug (ADC) conjugation comprising, adding D-α-Tocopheryl polyethylene glycol 1000 succinate (TPGS) to a formulation comprising an unconjugated antibody intermediate.

67. The method according to claim 66, wherein D-α-Tocopheryl polyethylene glycol 1000 succinate (TPGS) is added to the formulation in an amount ranging from about 0.001% to about 1%.
